(19) United States
(12) Patent Application Publication
(10) Pub. No.: US 2022/0023549 A1
(43) Pub. Date: Jan. 27, 2022

(54) Needle Safety Mechanisms

(71) Applicants: Fadra M. Whyte, (US); Ryan S. McPhee, (US)

(72) Inventors: Fadra M. Whyte, San Diego, CA (US); Ryan S. McPhee, San Diego, CA (US); Matt Roman, San Diego, CA (US)

(21) Appl. No.: 17/382,301

(22) Filed: Jul. 21, 2021

Related U.S. Application Data
(60) Provisional application No. 63/055,246, filed on Jul. 22, 2020.

(51) Int. Cl. A61M 5/32

(52) U.S. Cl. CPC A61M 5/3257

(57) ABSTRACT

The principles disclosed herein are directed toward needle safety mechanisms in which a needle is mounted in a hub, and a spring-biased safety cap is coupled to the hub. When the safety cap is released from the hub, the spring extends along the length of the needle until the cap is positioned at or beyond the end of the needle. After a needle is used for a procedure, the safety cap is extended to prevent or reduce the chance of unwanted needle sticks.

### BACKGROUND

Injections are a common procedure in dental procedures. After the injection is completed, the needle remains exposed until it is secured in some manner. Existing methods include inserting the end of the needle back into the cap or into another safety device. Nonetheless, accidents occur. The needle can be accidentally stuck into a dentist's or assistant's hand, or it can be dropped while still exposed, in all cases presenting a risk of infection. Thus, there exists a need for improved safety among dental needles to reduce or eliminate needle sticks.

A conventional dental syringe is used with replaceable needles and sterilized after each use. At one end of the syringe is an interface to receive a dental needle. Conventional needles comprise a cannula and a hub, with a rear cap on the syringe end and a front cap on the insertion end. The hub is designed to be screwed onto the end of a syringe. In operation, a dentist or assistant removes the rear cap, inserts the syringe end of the needle into the syringe, and screws the hub onto the syringe. Then the front cap is removed, which exposes the insertion end for use in the dental procedure.

Unlike needles used in medical procedures more generally, dental needles tend to serve a particular purpose, namely to inject a solution into a patient's mouth. An injection into the limited space inside a person's mouth makes it challenging to incorporate side-mounted safety mechanisms seen in needles for other medical applications. Thus, there exists a need for a safety mechanism that produces little or no interference with intraoral injections.

Existing syringes are widely used, with dental offices stocking multiple syringes so that one may be used while others are being sterilized. There exists a need for a safety mechanism that can be provided with the needle and which would permit dentists to continue to use their existing syringes without further modification to the syringes.

### SUMMARY

Various embodiments of the principles disclosed herein are directed toward needle safety mechanisms in which a needle is mounted in a hub, and a spring-biased safety cap is coupled to the hub. When the safety cap is released from the hub, the spring extends along the length of the needle until the cap is positioned at or beyond the end of the needle. After a needle is used for a procedure, the safety cap is extended to prevent or reduce the chance of unwanted needle sticks. In some embodiments, telescoping cylinders are coupled to the hub. When released, the cylinders extend and enclose the front end of the needle. In some embodiments, one or more button arms are used to release the cap. In some embodiments, a user squeezes the hub in order to release the safety cap.

### BRIEF DESCRIPTION OF THE DRAWINGS

The detailed description makes reference to the accompanying figures wherein:

FIG. 1 illustrates a side view of a prior art needle.

FIG. 2 illustrates a side view of an embodiment of a needle safety mechanism.

FIG. 3A illustrates a side view of an embodiment of a protective end cap for the configuration shown in FIG. 2.

FIG. 3B illustrates an end view of the protective end cap shown in FIG. 3A.

FIG. 3C illustrates a side view of a needle safety mechanism with a protective end cap as shown in FIGS. 3A and 3B.

FIG. 4 illustrates a side view of a syringe with a needle safety mechanism as disclosed with reference to FIG. 3C.

FIG. 5A illustrates a side view of a telescoping needle safety mechanism with two release arms.

FIG. 5B illustrates a side view of a telescoping needle safety mechanism after the cap has been released.

FIG. 6A illustrates a side view of an embodiment of a protective end cap.

FIG. 6B illustrates an end view of the protective end cap shown in FIG. 6A.

FIG. 6C illustrates a side view of a needle safety mechanism with a protective end cap as shown in FIGS. 6A and 6B.

FIG. 6D illustrates a side view of a needle safety mechanism with a protective end cap as shown in FIGS. 6A and 6B.

FIG. 7 illustrates a side view of a needle safety mechanism with a sheath.

FIG. 8A illustrates a side view of an embodiment of a protective end cap with a needle passing through it.

FIG. 8B illustrates a side view of the protective end cap shown in FIG. 8A after it has extended past the end of a needle.

FIG. 9A illustrates a side view of an embodiment of a protective end cap with a needle passing through it.

FIG. 9B illustrates a side view of the protective end cap shown in FIG. 9A after it has extended past the end of a needle.

FIG. 10A illustrates a side view of an embodiment of a protective end cap with a needle passing through it.

FIG. 10B illustrates a side view of the protective end cap shown in FIG. 10A after it has extended past the end of a needle.

FIG. 11A illustrates a side view of an embodiment of a protective end cap with a needle passing through it.

FIG. 11B illustrates a side view of the protective end cap shown in FIG. 11A after it has extended past the end of a needle.

FIG. 12A illustrates a side view of an embodiment of a protective end cap with a needle passing through it.

FIG. 12B illustrates a side view of the protective end cap shown in FIG. 12A after it has extended past the end of a needle.

FIG. 13A illustrates a telescoping needle safety mechanism with fins on the exterior surfaces of some cylinders to prevent the telescoping mechanism from retracting once extended.

FIG. 13B illustrates a telescoping needle safety mechanism with cylinders as shown in FIG. 13A in a retracted position with caps around the needle.

FIG. 13C illustrates a telescoping needle safety mechanism with cylinders as shown in FIGS. 13A and 13B in a retracted position.

FIG. 13D illustrates a telescoping needle safety mechanism with cylinders as shown in FIGS. 13A-13C in an extended position.

FIG. 14A illustrates a telescoping needle safety mechanism with fins on the interior surfaces of some cylinders to prevent the telescoping mechanism from retracting once extended.

FIG. 14B illustrates a telescoping needle safety mechanism with cylinders as shown in FIG. 14A in a retracted position.

FIG. 14C illustrates a telescoping needle safety mechanism with cylinders as shown in FIGS. 14A-14B in an extended position.

FIG. 15A illustrates a telescoping needle safety mechanism with fins formed in the surface of some cylinders to prevent the telescoping mechanism from retracting once extended.

FIG. 15B illustrates a telescoping needle safety mechanism with cylinders as shown in FIG. 15A in a retracted position.

FIG. 15C illustrates a telescoping needle safety mechanism with cylinders as shown in FIGS. 15A-15B in an extended position.

FIG. 16 illustrates a side view of a needle safety mechanism with an elongated hub.

FIG. 17A illustrates a side view of an embodiment of a needle safety mechanism with a button arm and a release arm.

FIG. 17B illustrates a side view of the needle safety mechanism in FIG. 17A without the spring.

FIG. 18A illustrates a side view of an embodiment of a needle safety mechanism with two button arms and two release arms.

FIG. 18B illustrates a side view of the needle safety mechanism in FIG. 18A without the spring.

FIG. 19A illustrates a side view of an embodiment of a needle safety mechanism with a spring biased release arm.

FIG. 19B illustrates a side view of the needle safety mechanism in FIG. 19A in the release position.

FIG. 20 illustrates a side view of an embodiment of a needle safety mechanism with a spring biased release arm.

FIG. 21 illustrates a side view of an embodiment of a needle safety mechanism with a spring biased release arm.

FIG. 22 illustrates a side view of an embodiment of a needle safety mechanism with a spring biased release arm.

FIG. 23 illustrates a side view of an embodiment of a needle safety mechanism with a squeeze release.

FIG. 24A illustrates an embodiment of a needle safety mechanism with a release frame to releasably secure a spring-loaded cap.

FIG. 24B illustrates a side view of the needle safety mechanism in FIG. 24A with the spring in a compressed position.

FIG. 24C illustrates a side view of the needle safety mechanism in FIGS. 24A and 24B with the spring in an extended position.

FIG. 25A illustrates a side view of an embodiment of a needle safety mechanism with a lever.

FIG. 25B illustrates a side view of the needle safety mechanism in FIG. 25A with the spring in a compressed position.

FIG. 25C illustrates a side view of the needle safety mechanism in FIGS. 25A and 25B with the spring in an extended position.

The figures are only intended to facilitate the description of the principles disclosed herein. The figures do not illustrate every aspect of the principles disclosed herein and do not limit the scope of the principles disclosed herein. Other objects, features, and characteristics will become more apparent upon consideration of the following detailed description.

### DETAILED DESCRIPTION

A detailed illustration is disclosed herein. However, techniques, methods, processes, systems and operating structures in accordance with the principles disclosed herein may be embodied in a wide variety of forms and modes, some of which may be quite different from those disclosed herein. Consequently, the specific structural and functional details disclosed herein are merely representative.

None of the terms used herein, including “spring,” “cylinder,” “hub,” “mechanism,” and “cap” are meant to limit the application of the principles disclosed herein. Reference to a cylinder or cylindrical shape shall not preclude embodiments in which another shape is used, including conical, triangular, rectangular, and other shapes. The terms “first,” “second,” and the like may refer to different or identical objects. The foregoing terms are used to illustrate the principles disclosed herein and are not intended to be limiting. Other explicit and implicit definitions may also be included below.

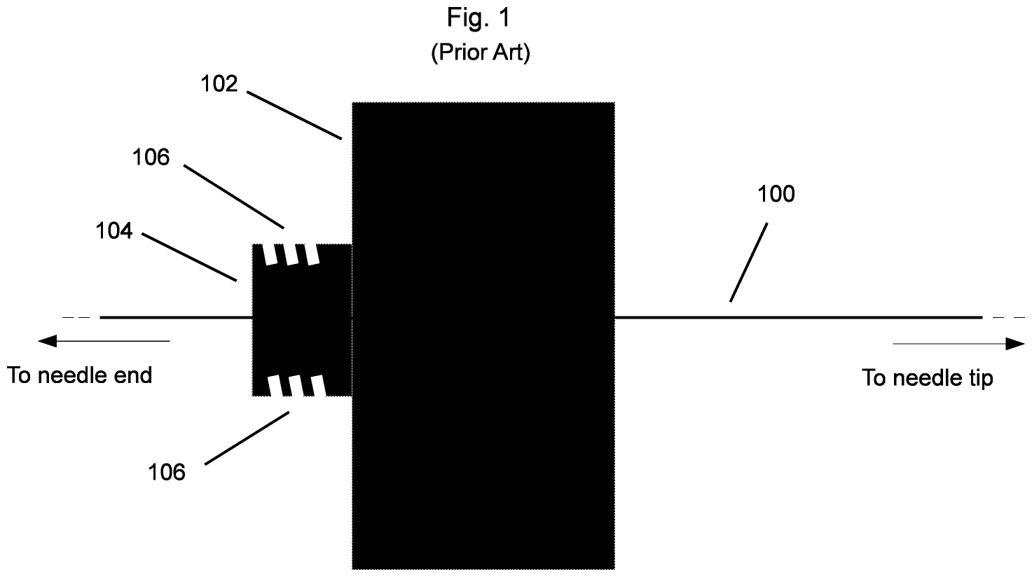

FIG. 1 depicts a side cross-section view of a conventional needle 100, with hub 102 and hub adapter 104. Hub adapter 104 comprises a threaded portion 106 configured to be screwed onto a syringe.

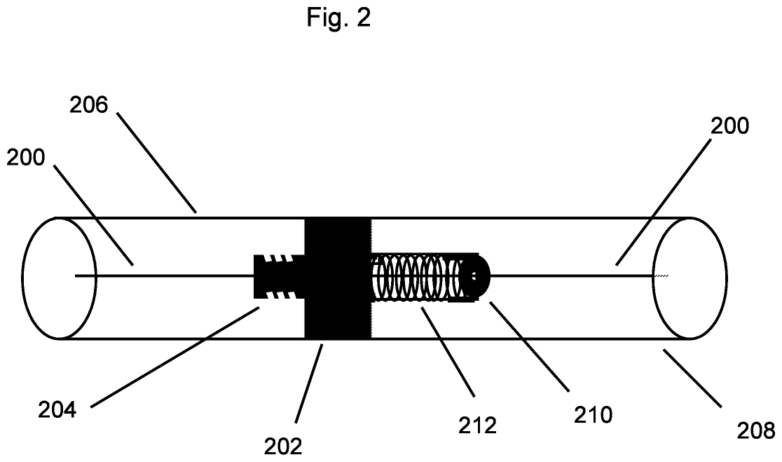

FIG. 2 depicts a side cross-section view of a needle comprising a safety mechanism. A needle 200 is secured by hub 202, which comprises hub adapter 204. A spring 212 and a safety cap 210 are coupled to hub 202. Rear cap 206 and front cap 208 are removably coupled to hub 202 and enclose needle 200.

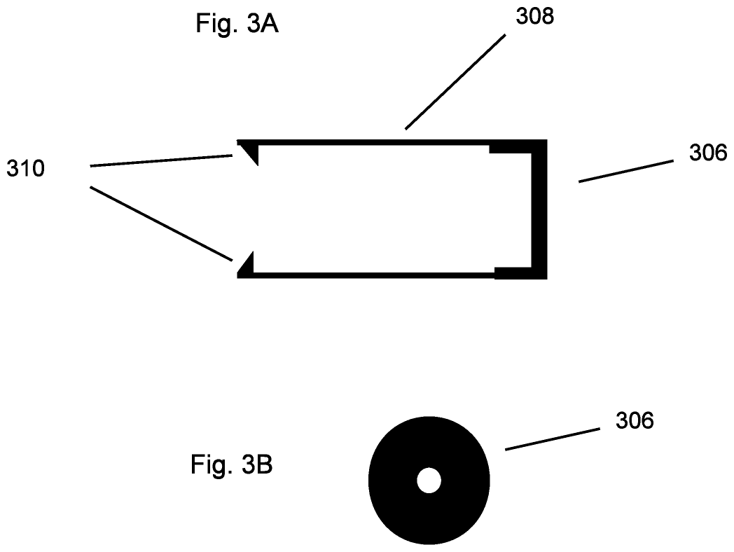

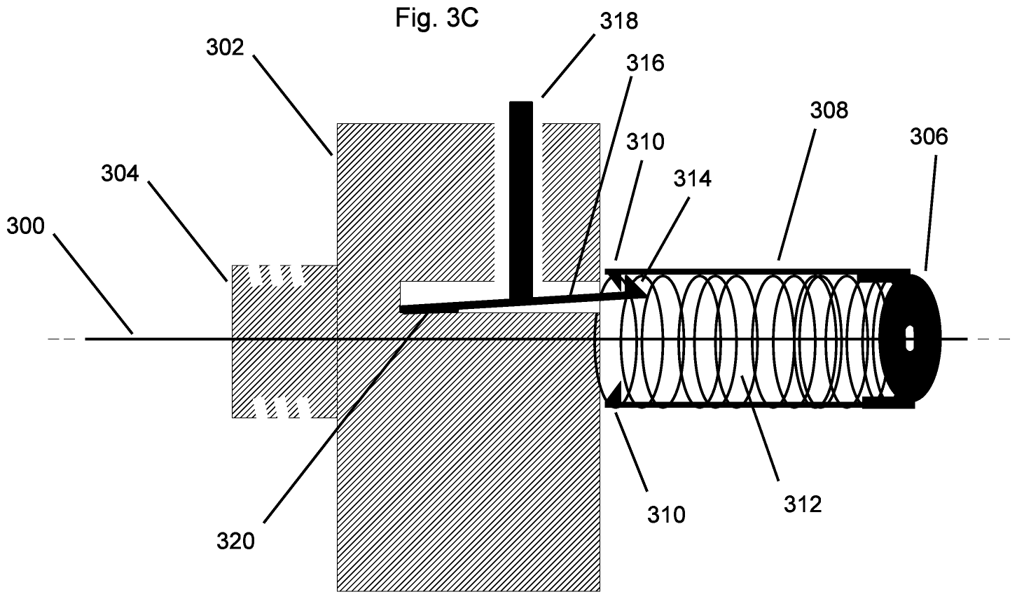

FIG. 3A depicts a side cross-section view of a safety cap 306 comprising sidewall 308 and cap retainer edge 310. It should be understood that safety cap 306 is generally cylindrical in shape, and sidewall 308 encloses a generally cylindrical area.

FIG. 3B depicts a front end view of safety cap 306. A hole is provided in the center for a needle to pass through.

FIG. 3C depicts a side cross-section view of a needle apparatus comprising a safety mechanism. To assist understanding, spring 312 and the front end of safety cap 306 are shown in perspective view. Needle 300 is secured by hub 302, which comprises hub adapter 304. A button arm 318 is coupled to a release arm 316, which is coupled to hub 302 at pivot point 320. At the end of release arm 316 is a release hook 314. When packaged for shipment and use, release hook 314 locks with cap retainer edge 310, thereby holding safety cap 306 in place. A spring 312 is compressed in the position shown. When button arm 318 is pressed, the downward force moves release arm 316 downward, which moves release hook 314 downward until it is clear of cap retainer edge 310. At that point, spring 312 pushes safety cap 306 to the right until the spring is extended to its natural state, preferably positioning the front end of safety cap 306 past the end of needle 300.

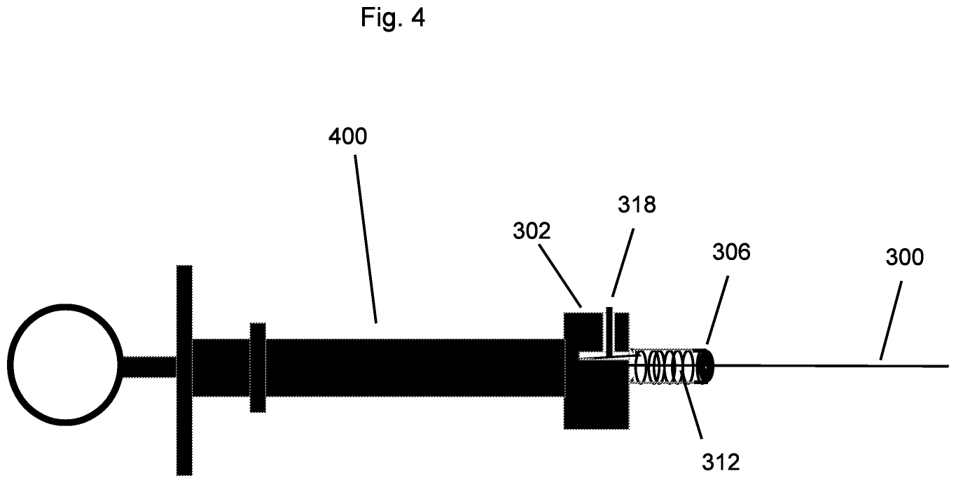

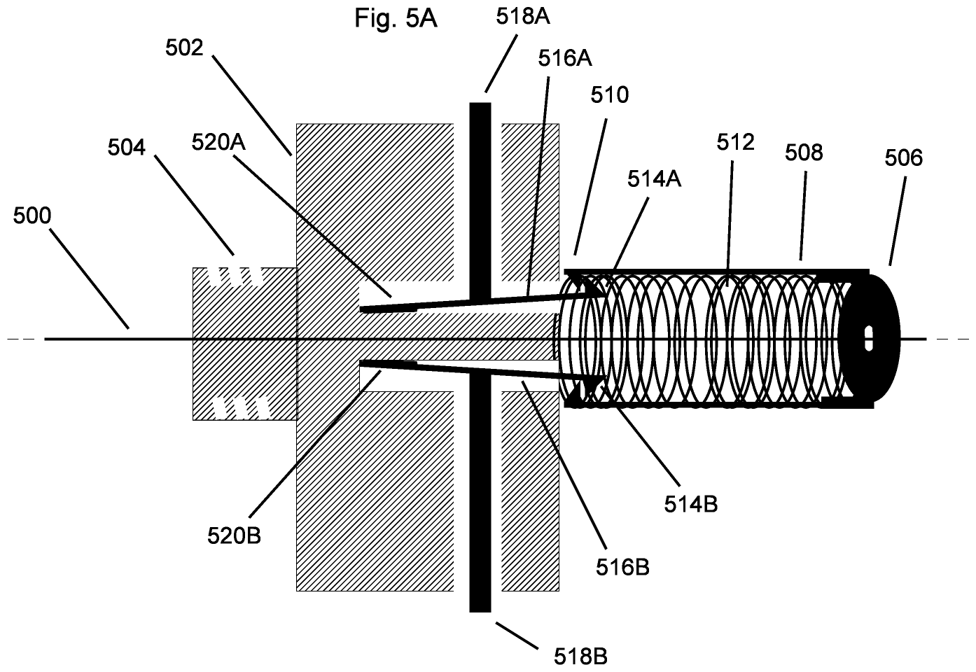

FIG. 4 depicts a side cross-section view of needle 300 mounted on a syringe 400. Hub adapter 304 (not shown) is screwed into the forward end of syringe 400. Button 318 arm is extended above the exterior of hub 302. Safety cap 306 is secured to hub 302, and spring 312 is in a compressed position.

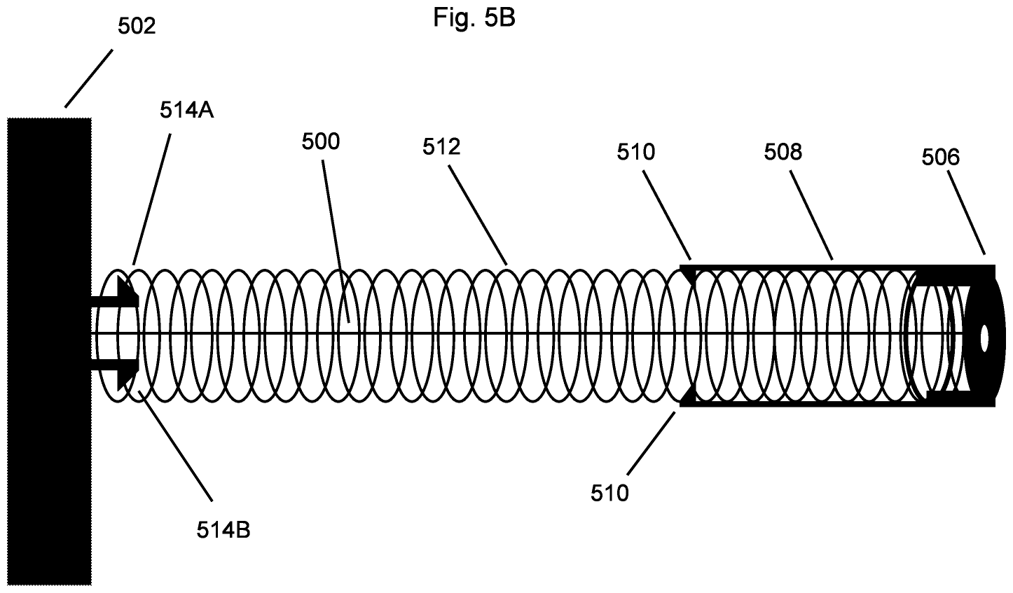

FIG. 5A depicts a side cross-section view of a needle apparatus comprising a safety mechanism with two button arms. To assist understanding, spring 512 and the front end of safety cap 506 are shown in perspective view. Needle 500 is secured by hub 302, which comprises hub adapter 504. A first button arm 518A is coupled to a first release arm 516A, which is coupled to hub 502 at first pivot point 520A. At the end of first release arm 516A is a first release hook 514A. A second button arm 518B is coupled to a second release arm 516B, which is coupled to hub 502 at second pivot point 520B. At the end of second release arm 516B is second release hook 514B. When packaged for shipment and use, first release hook 514A and second release hook 514B are locked with cap retainer edge 510, thereby holding safety cap 506 in place. A spring 512 is compressed in the position shown. When first button arm 518A and second button arm 518B are pressed, the inward forces move first release arm 516A downward and second release arm 516B upward, which moves first release hook 514A downward and second release hook 514B upward, until both are clear of cap retainer edge 510. At that point, spring 512 expands towards its natural state, pushing safety cap 506 to the right.

FIG. 5B depicts a side cross-section view of safety cap 506 after it is released and spring 512 has extended towards its natural state. Preferably, the front end of safety cap 506 is positioned past the end of needle 500 when spring 500 comes to rest.

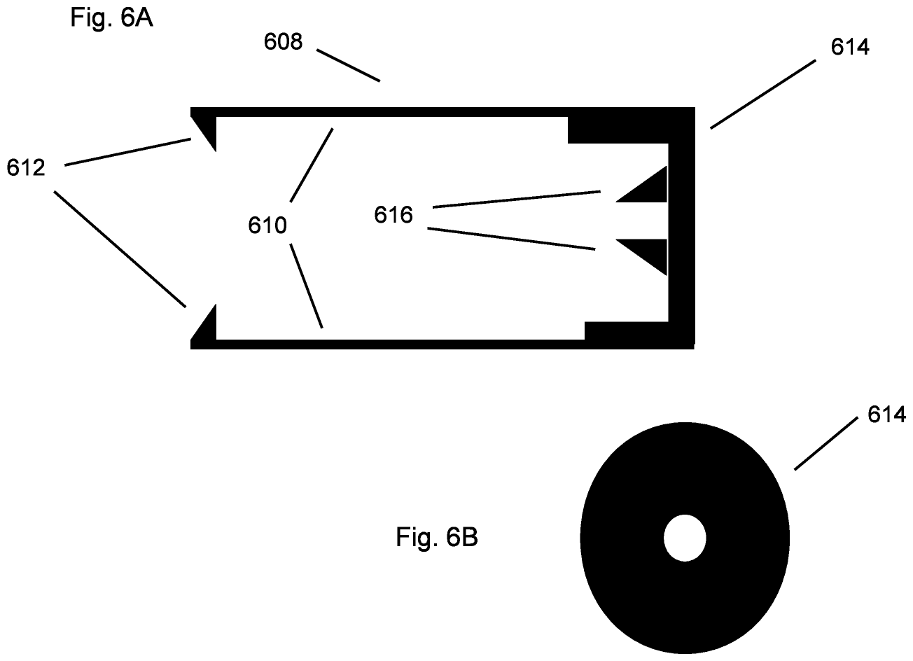

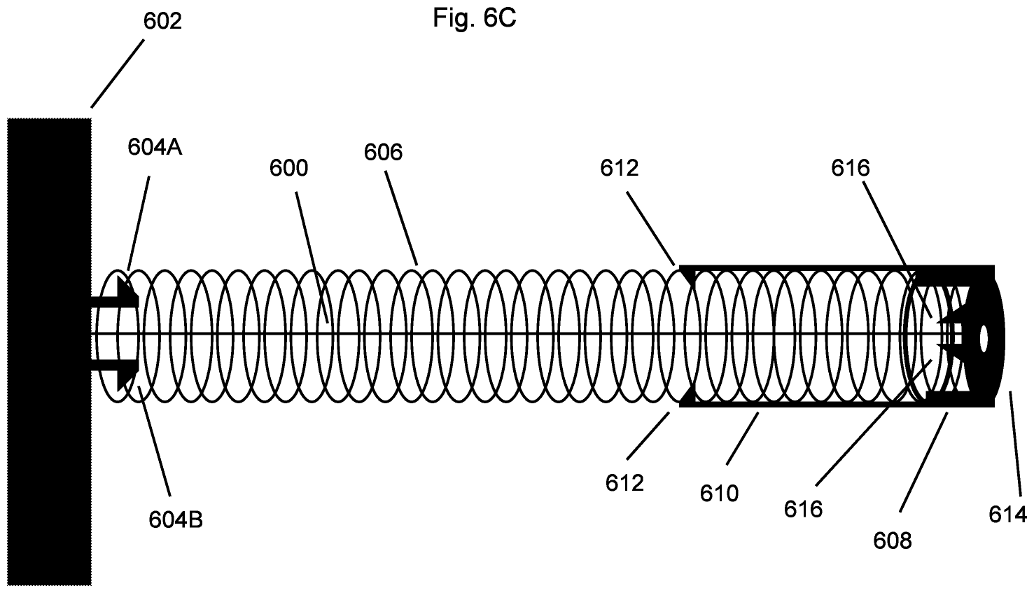

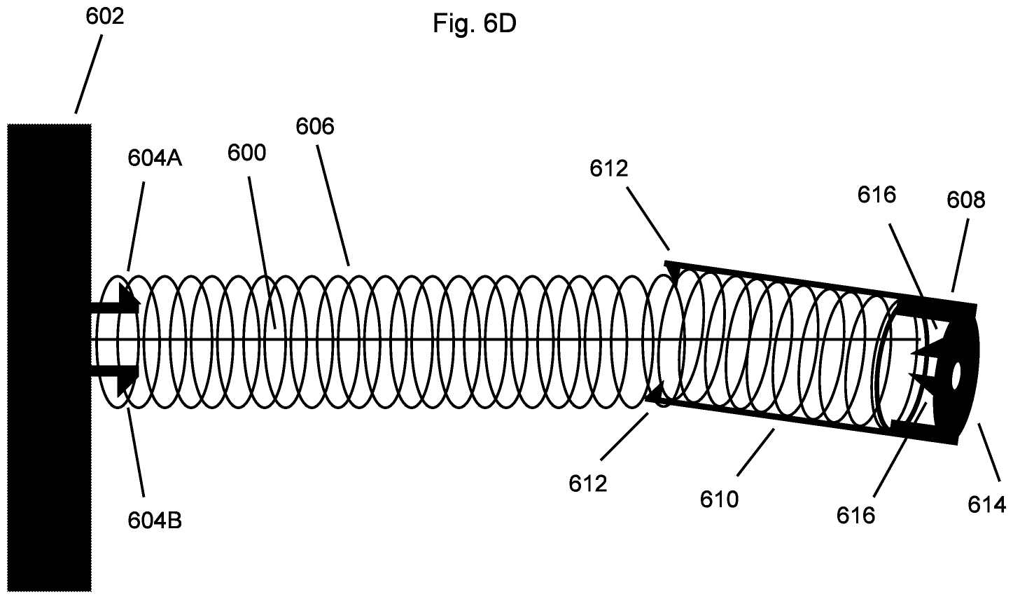

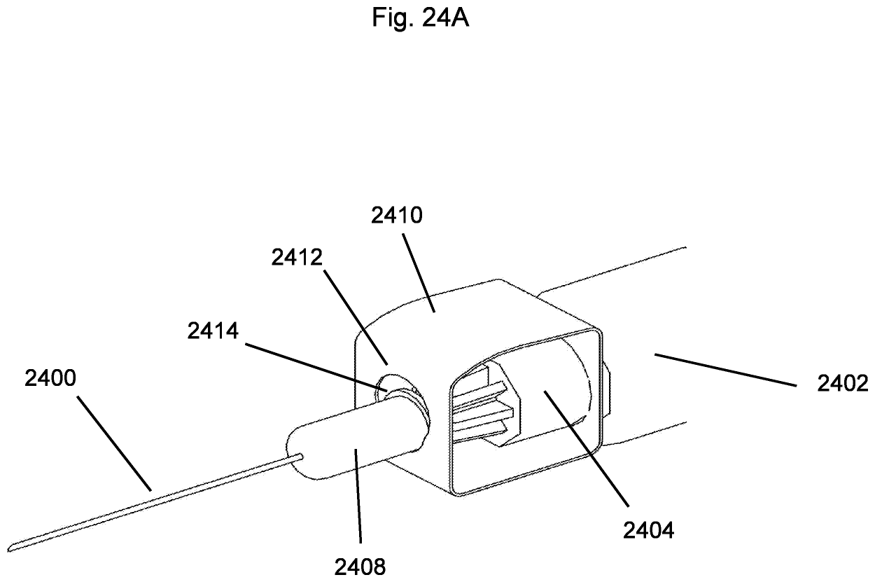

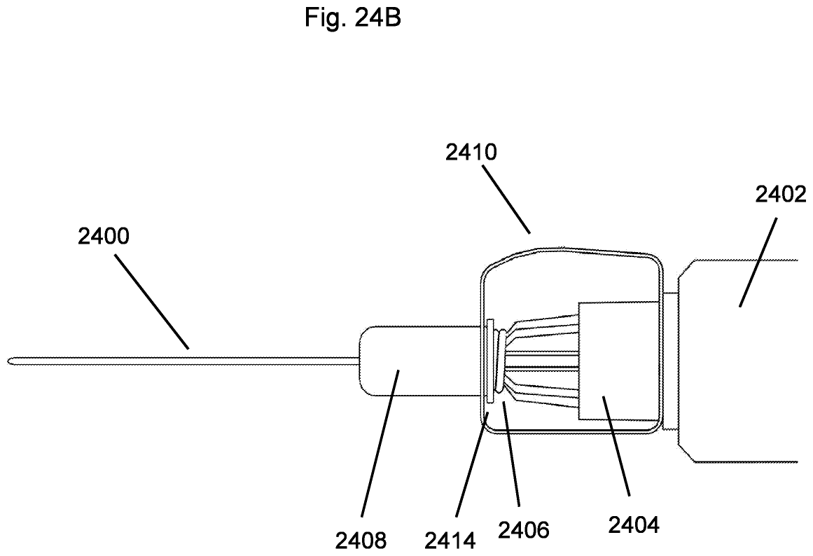

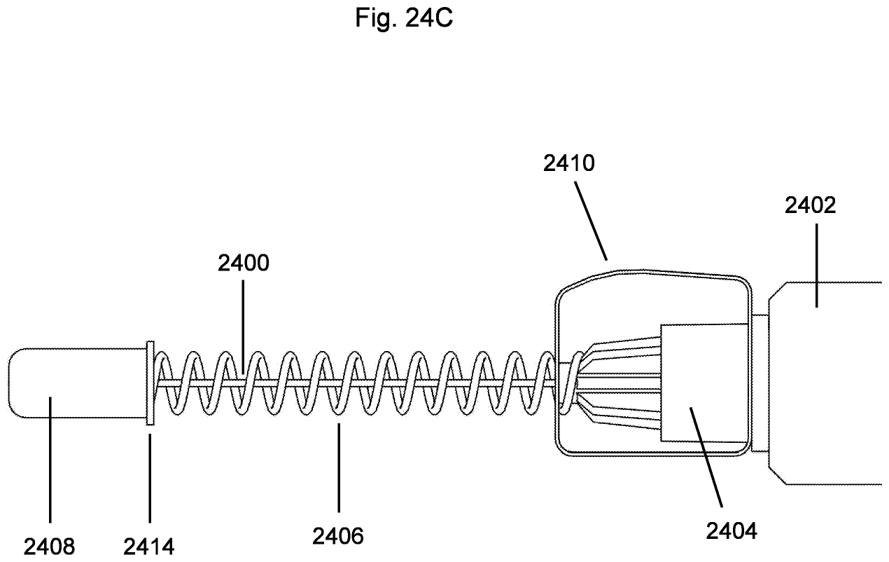

FIG. 6A depicts a side cross-section view of a safety cap with sidewall 610, cap retainer edge 612, cap disk 614, and cap interior protrusion 616 which is depicted as substantially cylindrical with a hole extending through it for a needle. In some embodiments, a safety cap retainer edge may be disposed on the exterior of the sidewall. Such a configuration enables the cap to be used with other release mechanisms, such as those described with reference to FIGS. 24A-C and 25A-C.

FIG. 6B depicts a front end view of cap disk 614. A hole is provided in the center for a needle to pass through.

FIG. 6C depicts a side cross-section view of safety cap 608 after it is released from hub 602 via movement of first release hook 604A and second release hook 604B. As shown, spring 606 has extended towards its natural state. Preferably, the cap disk 614 is positioned past the end of needle 600 when spring 606 comes to rest.

FIG. 6D depicts a side cross-section view of safety cap 608 in a position where spring 606 has extended the end of safety cap 608 past the end of needle 600 so that the end of needle 600 lies between cap interior protrusion and sidewall 610. In this state, the end of needle 600 is substantially or entirely surrounded by sidewall 610, cap disk 614, and cap interior protrusion 616. Thus, the end of needle 600 is secured from accidental needle sticks.

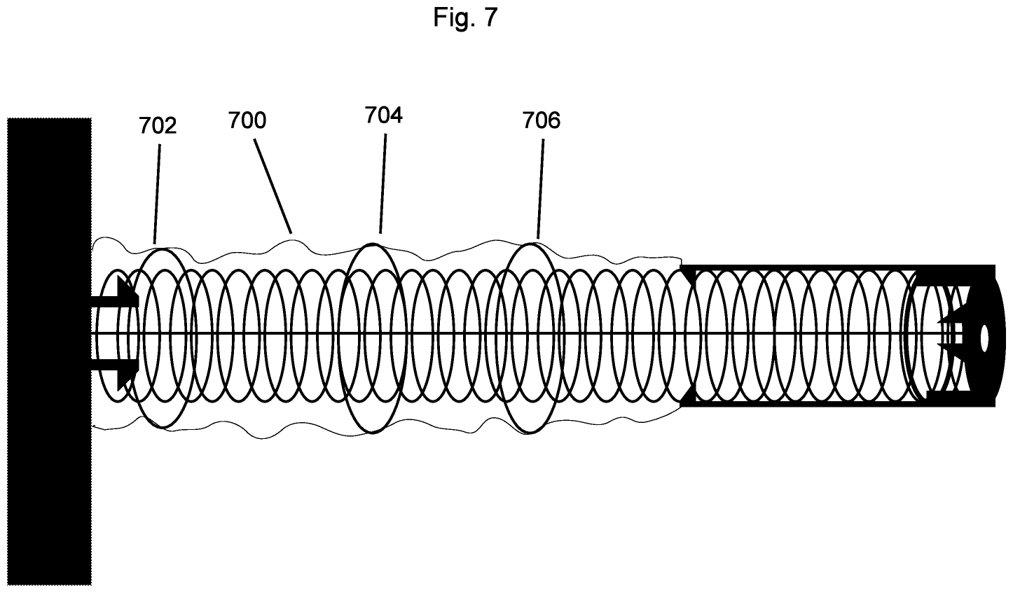

FIG. 7 depicts a side cross-section view of a safety cap and hub with an additional sheath 700 that encircles the spring and needle inside when the safety cap is released from the hub. Optional sheath rings 702, 704, and 706 may be used to assist in positioning sheath 700.

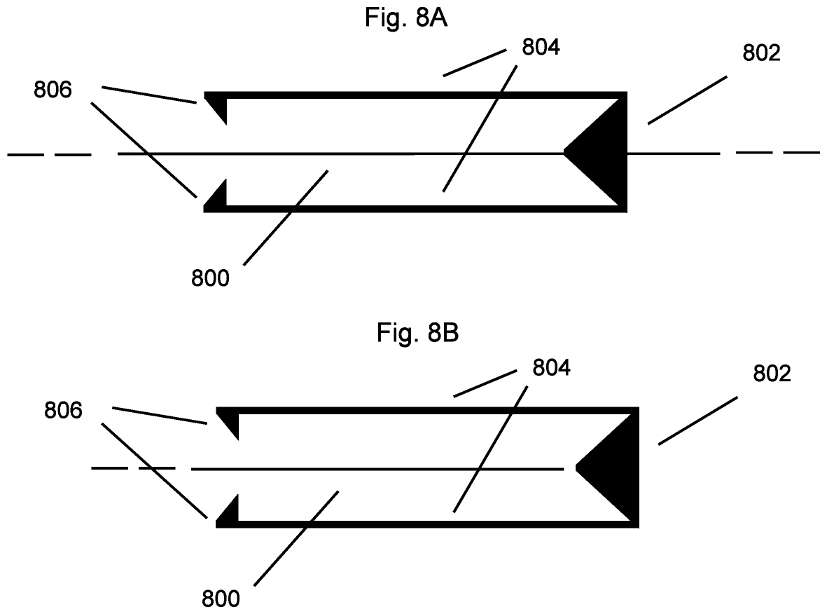

FIG. 8A depicts a side cross-section view of a safety cap with sidewall 804 and cap retainer edge 806 that encircle needle 800. At the front of the safety cap is a cone-shaped safety valve 802 which is configured to press lightly against needle 800 when the safety cap is in the retracted position and held in place by a hub.

FIG. 8B shows the safety cap after it has been moved past the end of needle 800. Once the forward end of needle 800 has passed through cone-shaped safety valve 802, the valve substantially or entirely closes so that the end of needle 800 cannot pass back through. Once in that position, the end of needle 800 is secured, preventing needle sticks.

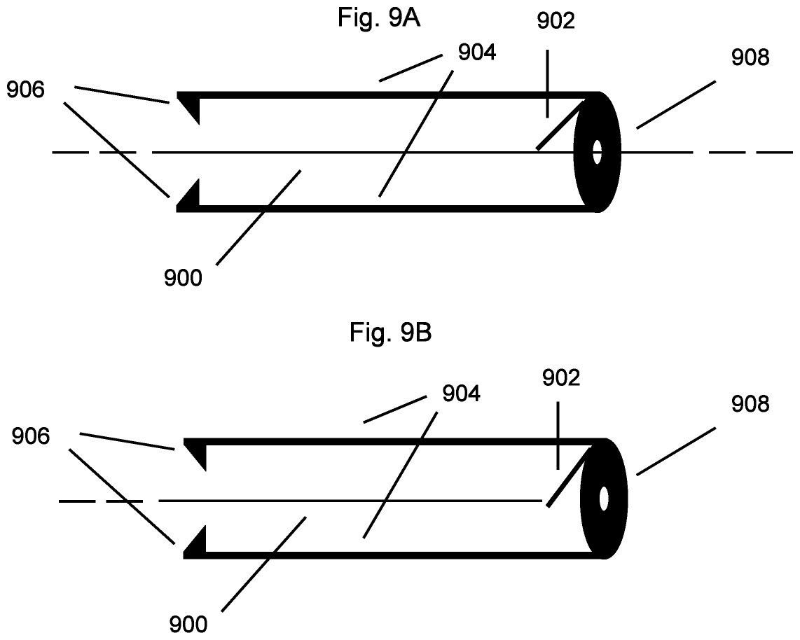

FIG. 9A depicts a side cross-section view of a safety cap with sidewall 904 and cap retainer edge 906 that encircle needle 900. At the front of the safety cap is an end cap disc 908 and a safety flap 902. End cap disc is shown in perspective view for ease of understanding. Safety flap 902 can be coupled to sidewall 904 and/or end cap disc 908 and is configured to press lightly against needle 900 when the safety cap is in the retracted position and held in place by a hub.

FIG. 9B shows the safety cap after it has been moved past the end of needle 900. Once the forward end of needle 900 has passed through end cap disc 908, safety flap 902 moves until it is positioned in front of the end of needle 900 so that the end of needle 900 cannot pass back through end cap disc 908. Once in that position, the end of needle 900 is secured, preventing needle sticks. Safety flap 902 may be configured in any suitable shape and size, with a cross-sectional area sufficient to block needle 900. Safety flap 902 comprises a material suitable to press against needle 900 without bending the needle or interfering with the delivery of medicine through the needle. Safety flap 902 further comprises a material that is suitable to slide along the side of needle 900 when the safety cap is released from the hub. Safety flap 902 further comprises a material that is capable of moving past the end of needle 900 at the appropriate time.

An alternative embodiment of the safety cap shown in FIGS. 9A and 9B omits end cap disc 908. In such an embodiment, operation of the safety cap remains the same. Once safety cap moves past the end of needle 900, safety flap 902 moves until it is positioned in front of the end of needle 900 so that the end of needle 900 cannot pass. The end of needle 900 is then secured between safety flap 902 and sidewall 904.

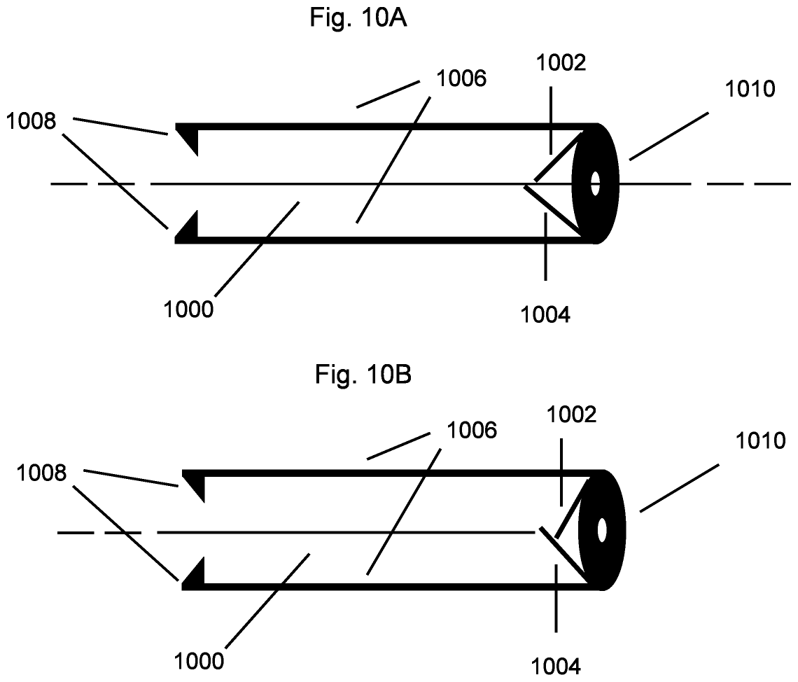

FIG. 10A depicts a side cross-section view of a safety cap with sidewall 1006 and cap retainer edge 1008 that encircle needle 1000. At the front of the safety cap is an end cap disc 1010 and a first safety flap 1002 and a second safety flap 1004. End cap disc is shown in perspective view for ease of understanding. First safety flap 1002 and second safety flap 1004 can each be coupled to sidewall 1006 and/or end cap disc 1010. First safety flap 1002 and second safety flap 1004 are each configured to press lightly against needle 1000 when the safety cap is in the retracted position and held in place by a hub.

FIG. 10B shows the safety cap after it has been moved past the end of needle 1000. Once the forward end of needle 1000 has passed through end cap disc 1010, first safety flap 1002 moves until it is positioned in front of the end of needle 1000. Second safety flap 1004 moves until it is positioned in front of end of needle 1000. In this position, needle 900 is secured by first safety flap 1002 and/or second safety flap 1004 and cannot pass back through end cap disc 1010. Once in that position, the end of needle 1000 is secured, preventing needle sticks. An alternative embodiment of the safety cap shown in FIGS. 10A and 10B omits end cap disc 1010. In such an embodiment, operation of the safety cap remains the same. Once safety cap moves past the end of needle 1000, first safety flap 1002 and second safety flap 1004 each moves until it is positioned in front of the end of needle 1000 so that the end of needle 1000 cannot pass. The end of needle 1000 is then secured between sidewall 1006 and either first safety flap 1002 or second safety flap 1004.

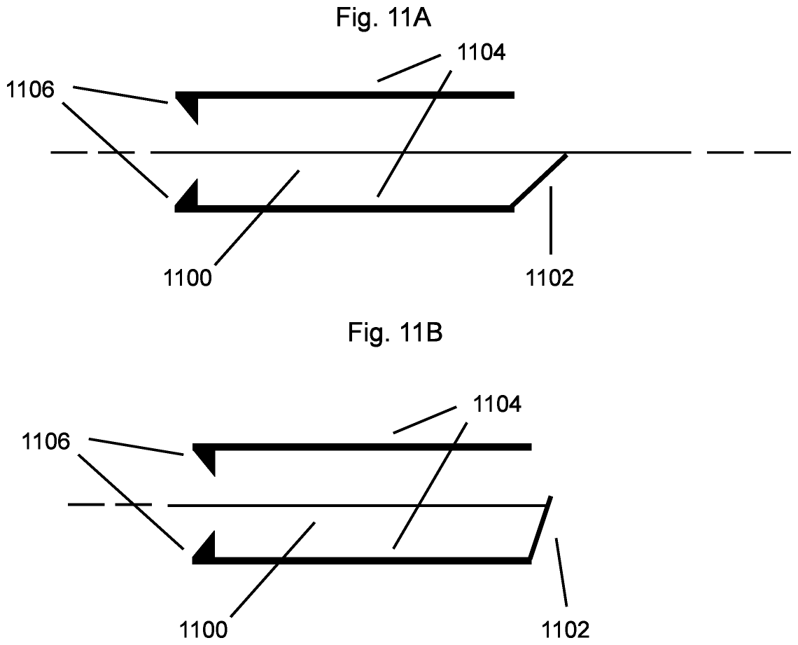

FIG. 11A depicts a side cross-section view of a safety cap with sidewall 1104 and cap retainer edge 1106 that encircle needle 1100. Towards the front of the safety cap is a safety flap 1102. Safety flap 1102 is coupled to sidewall 1104 and extends forward along the length of needle 1100. FIG. 11A depicts safety flap 1102 coupled to sidewall 1104 at the front end of sidewall 1104, but in alternative embodiments, safety flap 1102 may be coupled to sidewall 1104 at another position along the length of sidewall 1104. Safety flap 1102 is configured to press lightly against needle 1100 when the safety cap is in the retracted position and held in place by a hub.

FIG. 11B shows the safety cap after it has been moved past the end of needle 1100. Once the forward end of needle 1100 has passed safety flap 1102, the flap moves until it is positioned in front of the end of needle 1100. Once in that position, the end of needle 1100 is secured by safety flap 1102, preventing needle sticks.

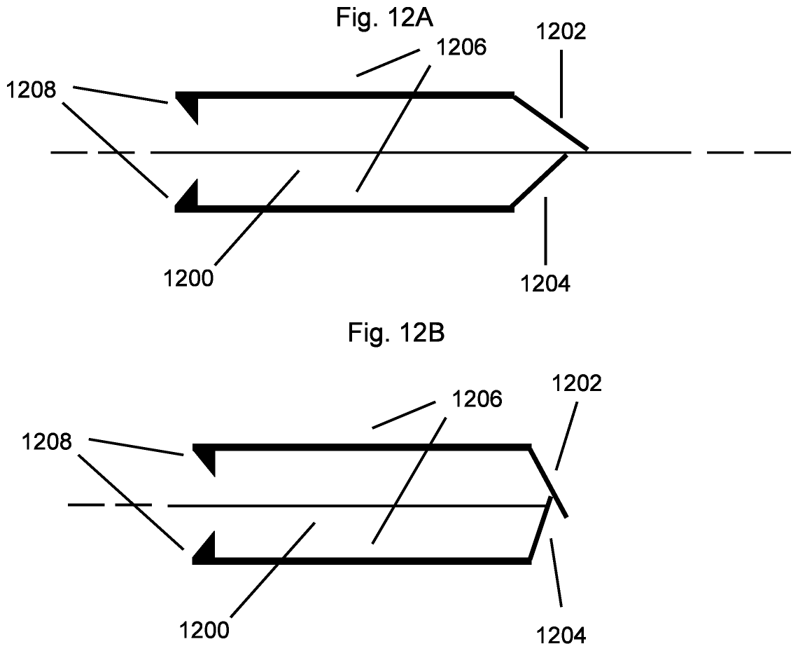

FIG. 12A depicts a side cross-section view of a safety cap with sidewall 1206 and cap retainer edge 1208 that encircle needle 1000. At the front of the safety cap is a first safety flap 1202 and a second safety flap 1204. First safety flap 1202 and second safety flap 1204 are each coupled to sidewall 1006. FIG. 12A depicts first safety flap 1202 and second safety flap 1204 coupled to sidewall 1206 at the front end of sidewall 1206, but in alternative embodiments, either or both of first safety flap 1202 and second safety flap 1204 may be coupled to sidewall 1206 at another position along the length of sidewall 1206. First safety flap 1202 and second safety flap 1204 are each configured to press lightly against needle 1200 when the safety cap is in the retracted position and held in place by a hub.

FIG. 12B shows the safety cap after it has been moved past the end of needle 1200. Once the forward end of needle 1200 has moved past first safety flap 1202, then first safety flap 1202 moves until it is positioned in front of the end of needle 1200. Second safety flap 1204 then moves until it is positioned in front of end of needle 1200. In this position, needle 1200 is secured by first safety flap 1202 and/or second safety flap 1204, preventing needle sticks.

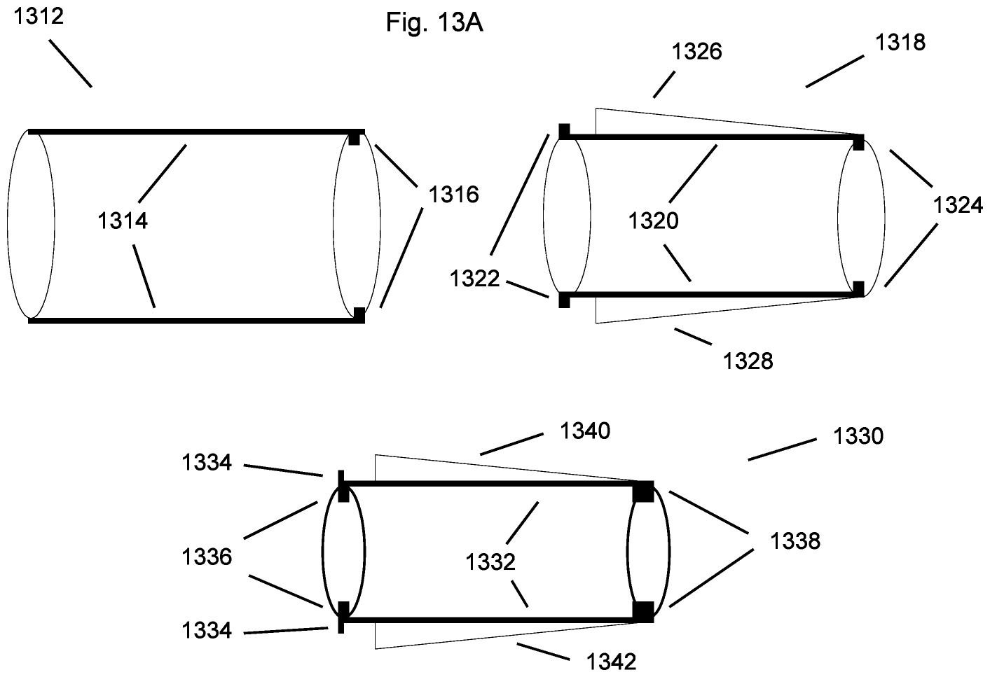

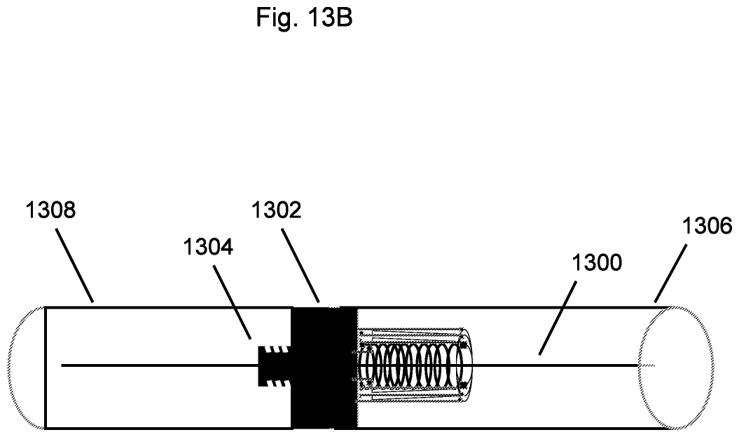

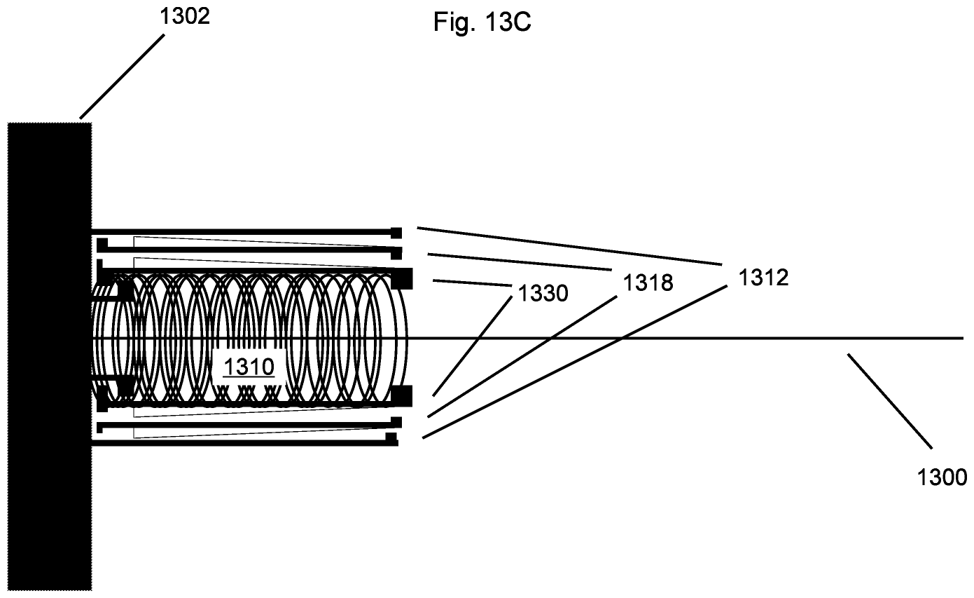

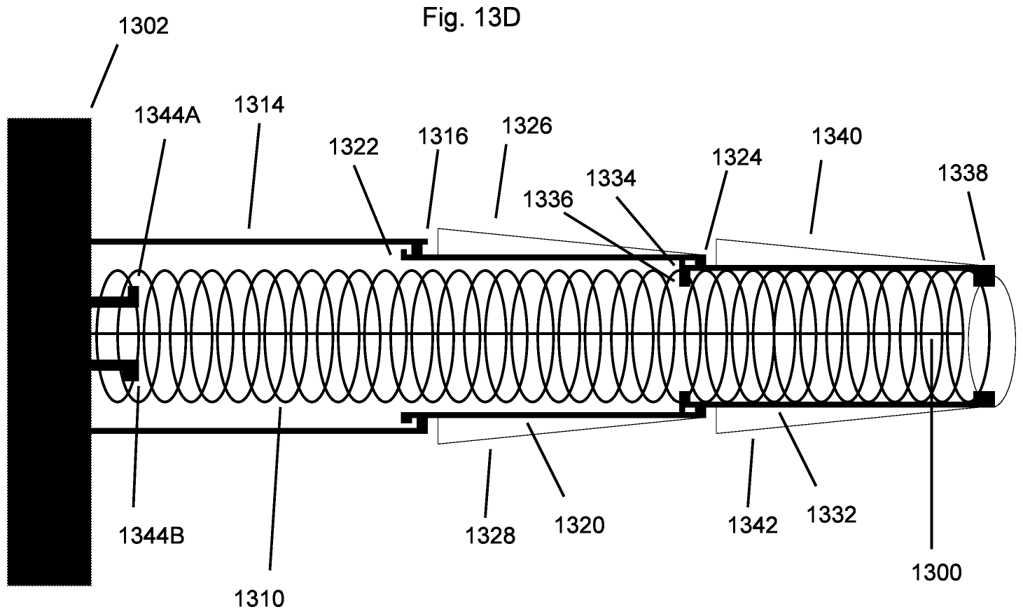

FIG. 13A shows three cylinders used for a safety mechanism. Outer cylinder 1312 comprises outer cylinder wall 1314 and outer cylinder lip 1316. Middle cylinder 1318 comprises middle cylinder wall 1320, middle cylinder rear lip 1322, and middle cylinder front lip 1324. Extending outward from middle cylinder wall 1320 is middle cylinder first fin 1326. Also shown is an optional middle cylinder second fin 1328. In other embodiments, additional middle cylinder fins may be disposed on middle cylinder wall 1320. Inner cylinder 1330 comprises inner cylinder wall 1332, inner cylinder rear lip 1334, inner cylinder retainer edge 1336, and an optional inner cylinder front lip 1338. Extending outward from inner cylinder wall 1332 is inner cylinder first fin 1340. Also shown is an optional inner cylinder second fin 1342. In other embodiments, additional inner cylinder fins may be disposed on inner cylinder wall 1332.

FIG. 13B depicts a side cross-section view of a needle comprising a safety mechanism comprising the elements depicted in FIG. 13A. Needle 1300 is secured by hub 1302, which comprises hub adapter 1304. Rear cap 1308 and front cap 1306 are removably coupled to hub 1302 and enclose needle 1300 and the safety mechanism.

FIG. 13C depicts a side cross-section view of a safety mechanism with telescoping cylinders in accordance with FIGS. 13A and 13B. To assist understanding, spring 1310 is shown in perspective view. When packaged for shipment and use, the front end of inner cylinder 1330 is coupled to the front end of spring 1310 and held in place by a release mechanism attached to hub 1302. Middle cylinder 1318 encircles inner cylinder 1330, and outer cylinder 1312 encircles middle cylinder 1318. Spring 1310 is compressed in the position shown. When the release mechanism is activated, spring 1310 expands towards its natural state, pushing inner cylinder 1330 to the right.

FIG. 13D depicts a side cross-section view of the safety mechanism after spring 1310 has extended towards its natural state. Inner cylinder 1330 is moved by spring 1310 to the right. As inner cylinder 1330 moves to the right, inner cylinder rear lip 1334 catches middle cylinder front lip 1324, pulling middle cylinder 1318 to the right. As middle cylinder 1318 moves to the right, middle cylinder rear lip 1322 catches outer cylinder lip 1316, thereby preventing middle cylinder 1318 from moving further to the right. At that point, middle cylinder front lip 1324 prevents inner cylinder rear lip 1334 from moving further, thereby preventing inner cylinder 1330 from moving further to the right. Once middle cylinder 1318 has extended sufficiently far, middle cylinder first fin 1326 and/or optional middle cylinder second fin 1328 extend outward from middle cylinder wall 1320. This prevents middle cylinder 1318 from retracting back within outer cylinder 1312, since middle cylinder first fin 1326 and/or optional middle cylinder second fin 1328 would extend outward past, and press against, outer cylinder wall 1314. Once inner cylinder 1330 has extended sufficiently far, inner cylinder first fin 1340 and/or optional inner cylinder second fin 1342 extend outward from inner cylinder wall 1332. This prevents inner cylinder 1330 from retracting back within middle cylinder 1318, since inner cylinder first fin 1340 and/or optional inner cylinder second fin 1342 would extend outward past, and press against, middle cylinder wall 1320. Preferably, the front end of inner cylinder 1330 is positioned past the end of needle 1300 when spring 1310 comes to rest.

The foregoing embodiment described with reference to FIGS. 13A-13D uses three telescoping cylinders. It should be understood that the principles disclosed herein may be used to create a safety mechanism with two cylinders, three cylinders, or four or more cylinders, without departing from the principles disclosed herein.

The telescoping cylinders described with reference to FIGS. 13A-13D and in other embodiments disclosed herein, are preferably configured in size, shape, and material so that they are held in place in the contracted position by friction. Specifically, in the contracted position, the middle cylinder (in a three-cylinder configuration) or middle cylinders (in a four or more cylinder configuration) are prevented from sliding forward by friction with the inner and/or outer cylinder (or other middle cylinders if applicable). Other means for securing the middle cylinder(s) may be employed without departing from the principles disclosed herein.

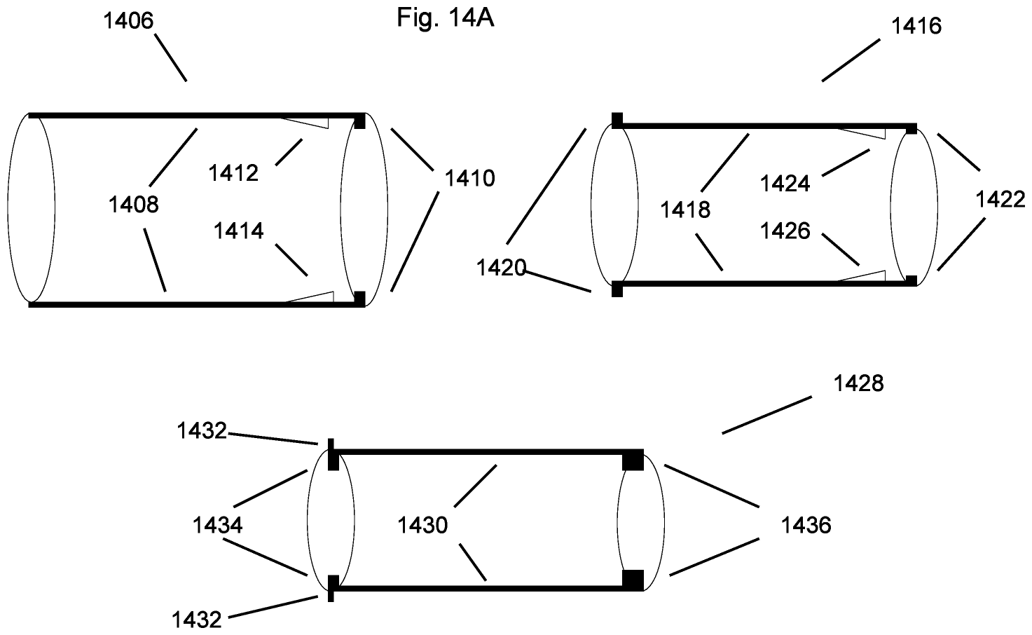

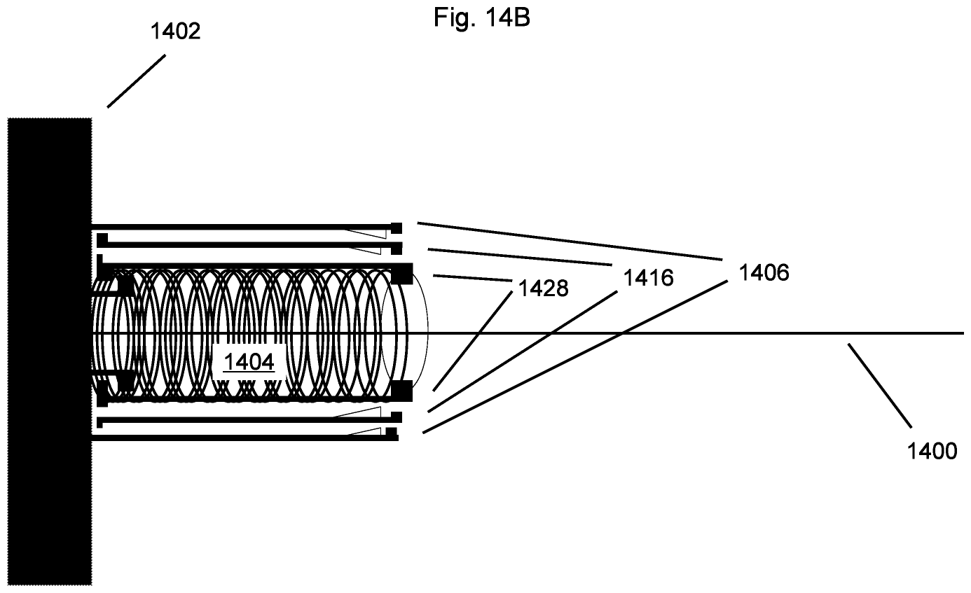

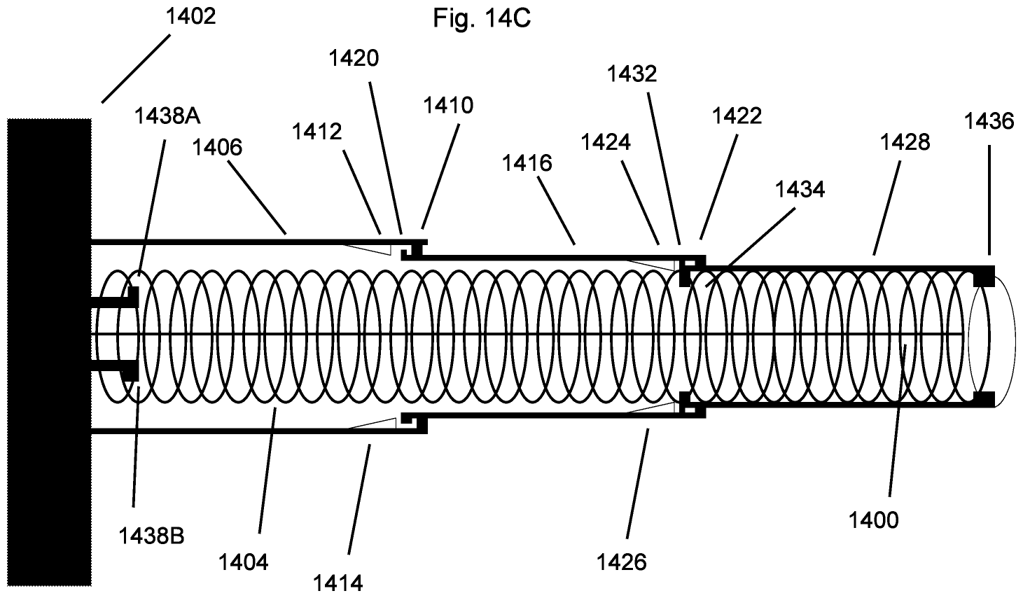

FIG. 14A shows three cylinders used for a safety mechanism. Outer cylinder 1406 comprises outer cylinder wall 1408 and outer cylinder lip 1410. Extending inward from outer cylinder wall 1408 is outer cylinder first interior fin 1412. Also shown is an optional outer cylinder second interior fin 1414. In other embodiments, additional outer cylinder interior fins may be disposed on outer cylinder wall 1408. Middle cylinder 1416 comprises middle cylinder wall 1418, middle cylinder rear lip 1420, and middle cylinder front lip 1422. Extending inward from middle cylinder wall 1418 is middle cylinder first interior fin 1424. Also shown is an optional middle cylinder second interior fin 1426. In other embodiments, additional middle cylinder interior fins may be disposed on middle cylinder wall 1418. Inner cylinder 1428 comprises inner cylinder wall 1430, inner cylinder rear lip 1432, inner cylinder retainer edge 1434, and an optional inner cylinder front lip 1436.

FIG. 14B depicts a side cross-section view of a needle comprising a safety mechanism comprising the elements depicted in FIG. 14A. Needle 1400 is secured by hub 1402. To assist understanding, spring 1404 is shown in perspective view. When packaged for shipment and use, the front end of inner cylinder 1428 is coupled to the front end of spring 1404 and held in place by a release mechanism attached to hub 1402. Middle cylinder 1416 encircles inner cylinder 1428, and outer cylinder 1406 encircles middle cylinder 1416. Spring 1404 is compressed in the position shown. When the release mechanism is activated, spring 1404 expands towards its natural state, pushing inner cylinder 1428 to the right.

FIG. 14C depicts a side cross-section view of the safety mechanism after spring 1404 has extended towards its natural state. Inner cylinder 1428 is moved by spring 1404 to the right. As inner cylinder 1428 moves to the right, inner cylinder rear lip 1432 catches middle cylinder front lip 1422, pulling middle cylinder 1416 to the right. As middle cylinder 1416 moves to the right, middle cylinder rear lip 1420 catches outer cylinder lip 1410, thereby preventing middle cylinder 1416 from moving further to the right. At that point, middle cylinder front lip 1422 prevents inner cylinder rear lip 1432 from moving further, thereby preventing inner cylinder 1428 from moving further to the right. Once middle cylinder 1416 has extended sufficiently far, outer cylinder first interior fin 1412 and/or optional outer cylinder second interior fin 1414 extend inward from outer cylinder wall 1408. This prevents middle cylinder 1416 from retracting back within outer cylinder 1406, since middle cylinder rear lip 1420 would extend outward past, and press against, the front edge of outer cylinder first interior fin 1412 and/or optional outer cylinder second interior fin 1414. Once inner cylinder 1428 has extended sufficiently far, middle cylinder first interior fin 1424 and/or optional middle cylinder second interior fin 1426 extend inward from middle cylinder wall 1418. This prevents inner cylinder 1428 from retracting back within middle cylinder 1416, since inner cylinder rear lip 1432 would extend outward past, and press against, the front edge of middle cylinder first interior fin 1424 and/or optional middle cylinder second interior fin 1426. Preferably, the front end of inner cylinder 1428 is positioned past the end of needle 1400 when spring 1404 comes to rest.

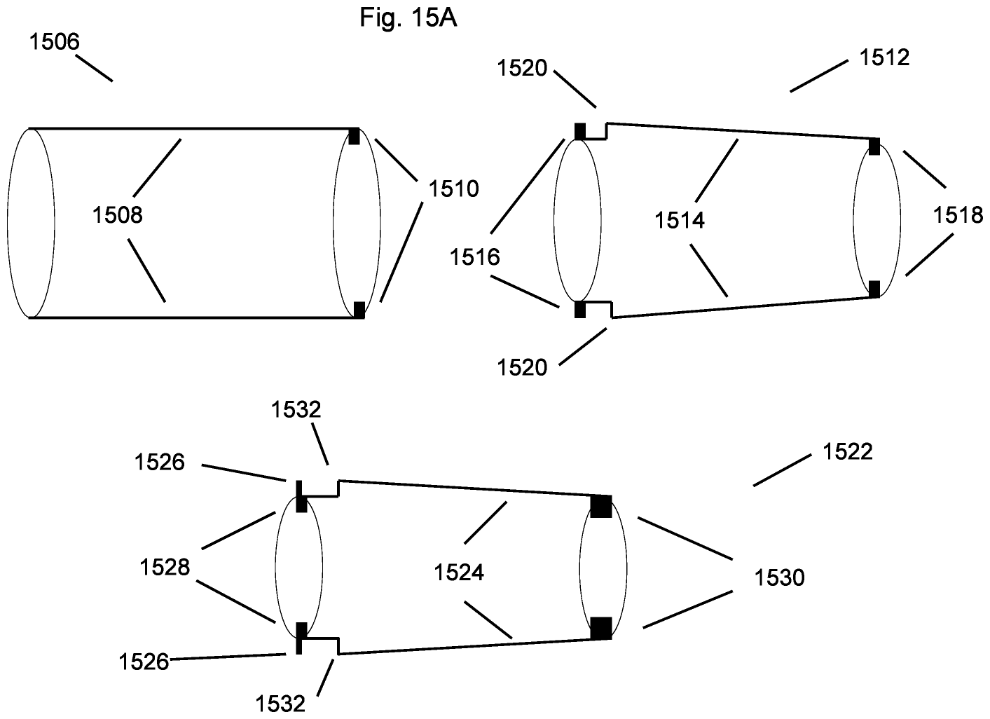

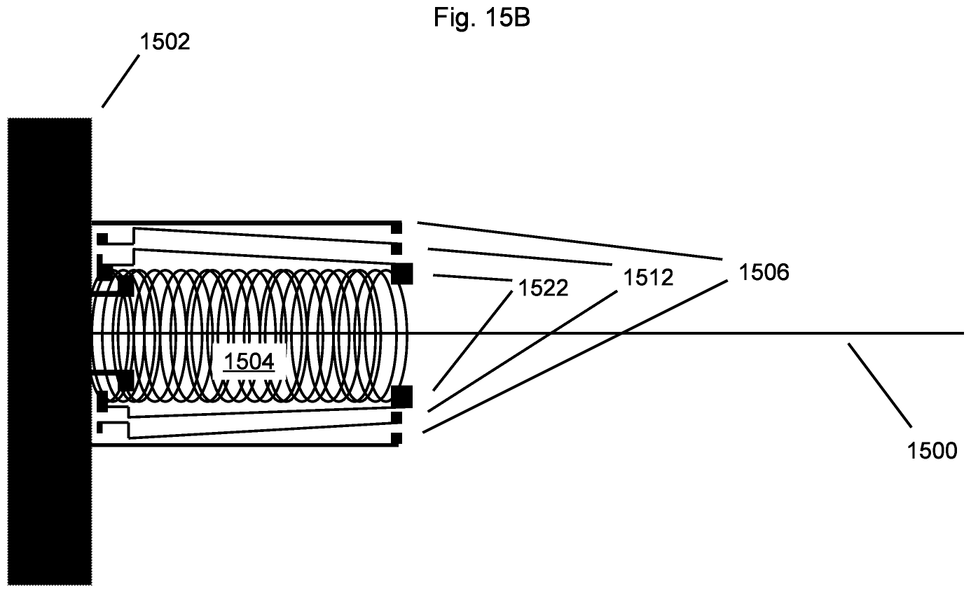

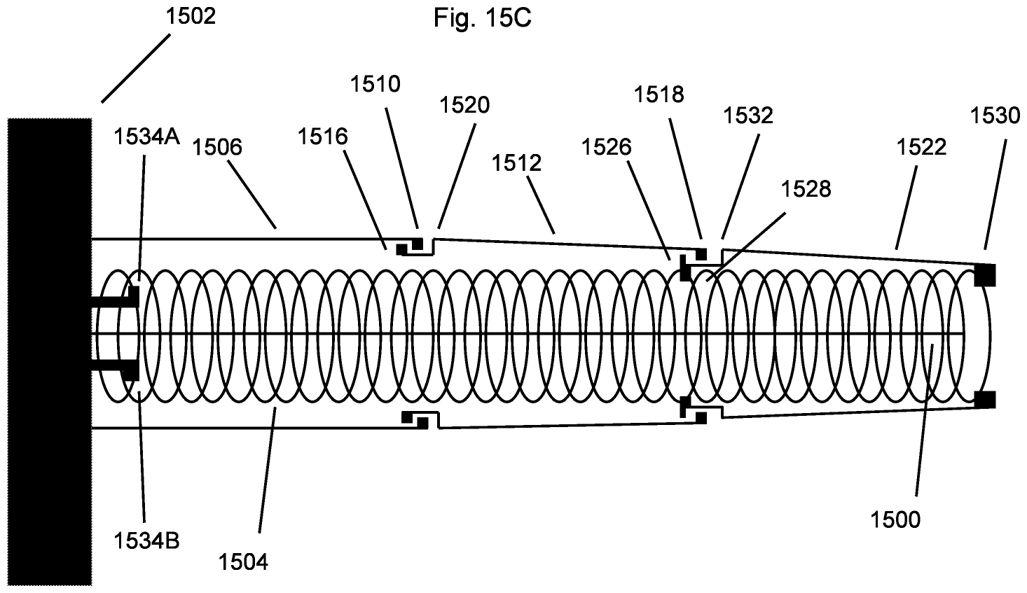

FIG. 15A shows three cylinders used for a safety mechanism. Outer cylinder 1506 comprises outer cylinder wall 1508 and outer cylinder lip 1510. Middle cylinder 1512 comprises middle cylinder wall 1514, middle cylinder rear lip 1516, and middle cylinder front lip 1518. Middle cylinder wall 1514 further comprises middle cylinder wall overhang 1520 substantially near the rear end of middle cylinder 1512 and substantially near middle cylinder rear lip 1516. Inner cylinder 1522 comprises inner cylinder wall 1524, inner cylinder rear lip 1526, inner cylinder retainer edge 1528, and an optional inner cylinder front lip 1530. Inner cylinder wall 1524 further comprises inner cylinder wall overhang 1532 substantially near the rear end of inner cylinder 1522 and substantially near inner cylinder rear lip 1526.

FIG. 15B depicts a side cross-section view of a needle comprising a safety mechanism comprising the elements depicted in FIG. 15A. Needle 1500 is secured by hub 1502. To assist understanding, spring 1504 is shown in perspective view. When packaged for shipment and use, the front end of inner cylinder 1522 is coupled to the front end of spring 1504 and held in place by a release mechanism attached to hub 1502. Middle cylinder 1512 encircles inner cylinder 1522, and outer cylinder 1506 encircles middle cylinder 1512. Spring 1504 is compressed in the position shown. When the release mechanism is activated, spring 1504 expands towards its natural state, pushing inner cylinder 1522 to the right.

FIG. 15C depicts a side cross-section view of the safety mechanism after spring 1504 has extended towards its natural state. Inner cylinder 1522 is moved by spring 1504 to the right. As inner cylinder 1522 moves to the right, inner cylinder rear lip 1526 catches middle cylinder front lip 1518, pulling middle cylinder 1512 to the right. As middle cylinder 1512 moves to the right, middle cylinder rear lip 1516 catches outer cylinder lip 1510, thereby preventing middle cylinder 1512 from moving further to the right. At that point, middle cylinder front lip 1518 prevents inner cylinder rear lip 1526 from moving further, thereby preventing inner cylinder 1522 from moving further to the right. Once middle cylinder 1512 has extended sufficiently far, outer cylinder lip 1510 is positioned between middle cylinder rear lip 1516 and middle cylinder wall overhang 1520. This prevents middle cylinder 1512 from retracting back within outer cylinder 1506, since middle cylinder wall overhang 1520 would extend outward past, and press against, outer cylinder lip 1510. Once inner cylinder 1522 has extended sufficiently far, middle cylinder front lip 1518 is positioned between inner cylinder rear lip 1526 and inner cylinder wall overhang 1532. This prevents inner cylinder 1522 from retracting back within middle cylinder 1512, since inner cylinder wall overhang 1532 would extend outward past, and press against, middle cylinder front lip 1518. Preferably, the front end of inner cylinder 1522 is positioned past the end of needle 1500 when spring 1504 comes to rest.

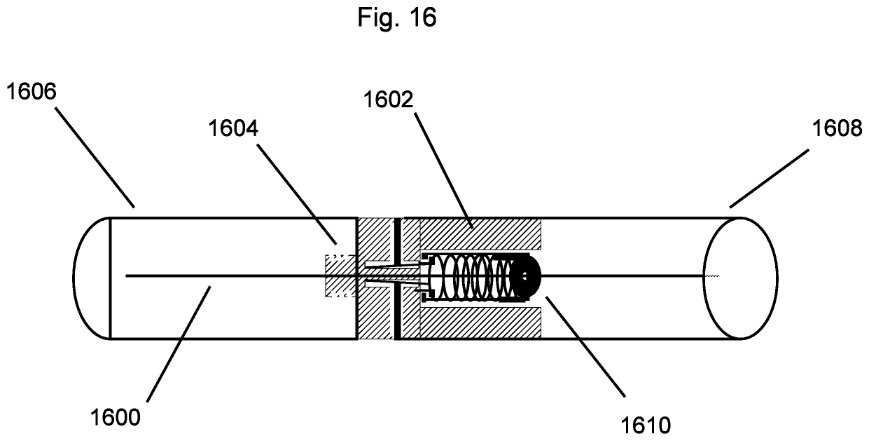

FIG. 16 depicts a side cross-section view of a needle comprising a safety mechanism with an elongated hub. Needle 1600 is secured by elongated hub 1602, which comprises hub adapter 1604. Rear cap 1606 and front cap 1608 are removably coupled to hub 1062 and enclose needle 1600 and the safety mechanism.

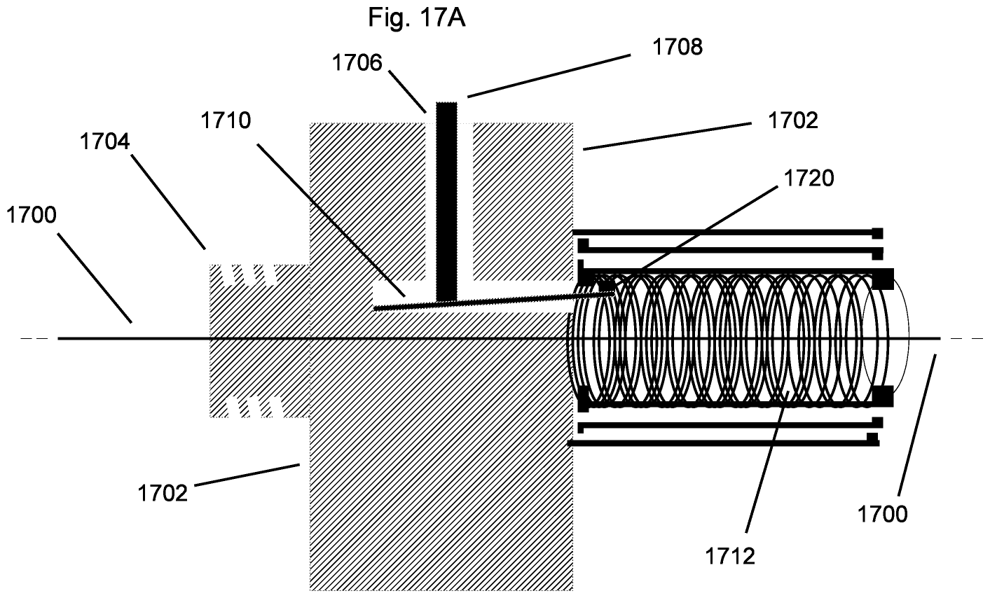

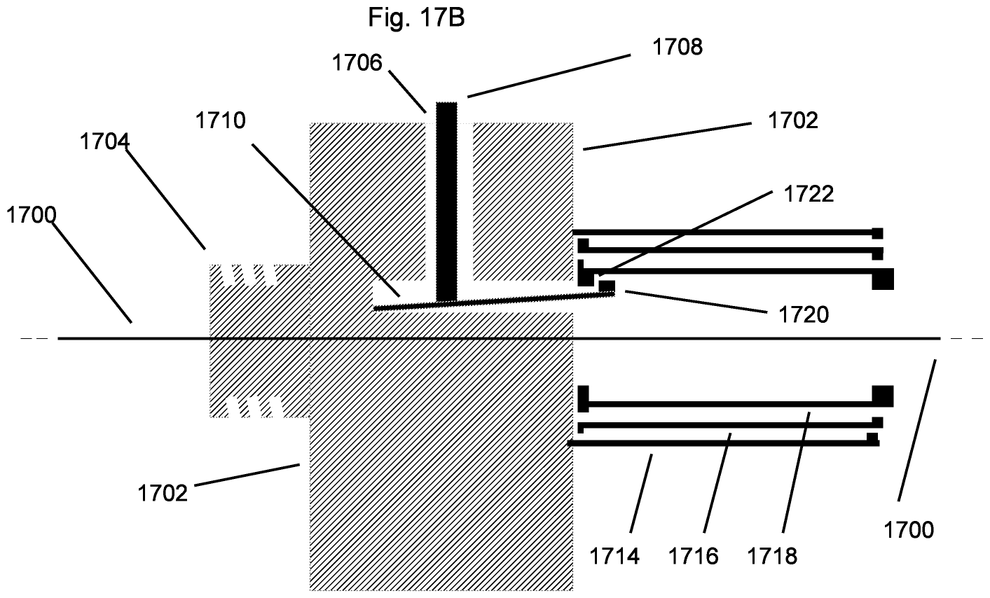

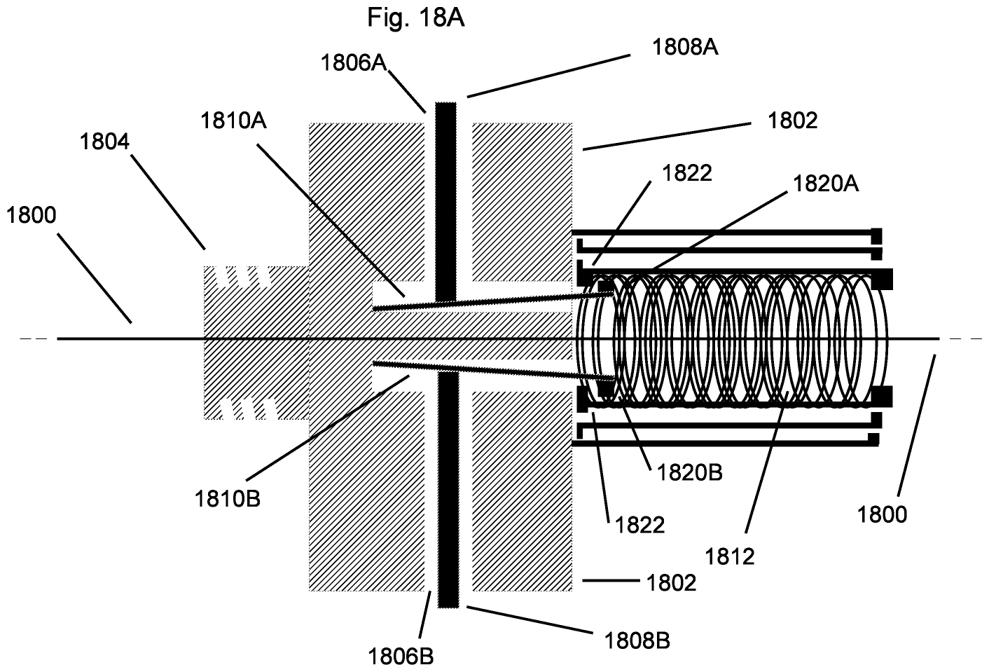

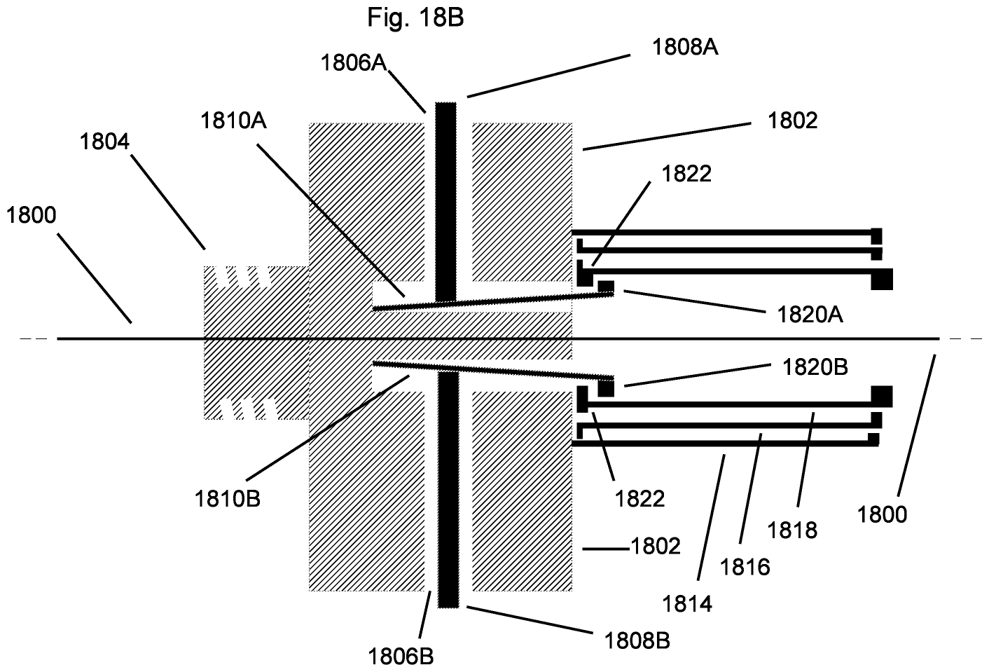

FIGS. 17A and 17B depict a side cross-section view of a needle apparatus comprising a safety mechanism incorporating a biased release arm. To assist understanding, spring 1712 is shown in perspective view in FIG. 17A and is removed from view in FIG. 17B. Needle 1700 is secured by hub 1702, which comprises hub adapter 1704. A button arm 1708 is positioned in button channel 1706 and is coupled to a release arm 1710, which is coupled to hub 1702 at or near one end of release arm 1710. At the opposite end of release arm 1710 is a release mechanism 1720. When packaged for shipment and use, release arm 1710 is biased upward so that release mechanism 1720 locks with cap retainer edge 1722, thereby holding inner cylinder 1718 in place. The bias may be obtained by the selection of material(s) for the release arm 1710 and hub 1702 and the design of the release arm and its connection to hub 1702. A spring 1712 is compressed in the position shown. In this position, inner cylinder 1718, middle cylinder 1716, and outer cylinder 1714 are held in place. When button arm 1708 is pressed, the downward force moves release arm 1710 downward, which moves release mechanism 1720 downward until it is clear of cap retainer edge 1722. At that point, spring 712 expands towards its natural state, pushing inner cylinder 1718 to the right.

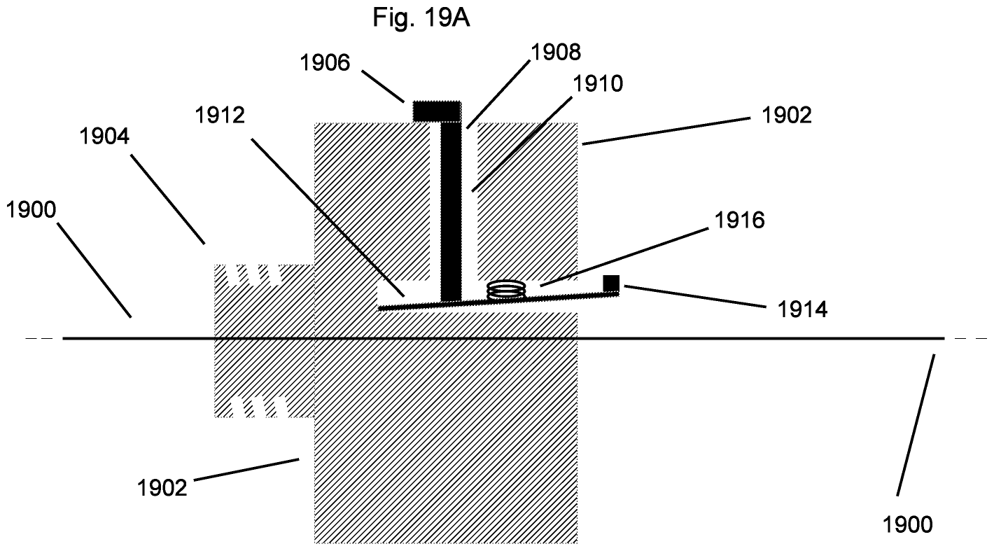

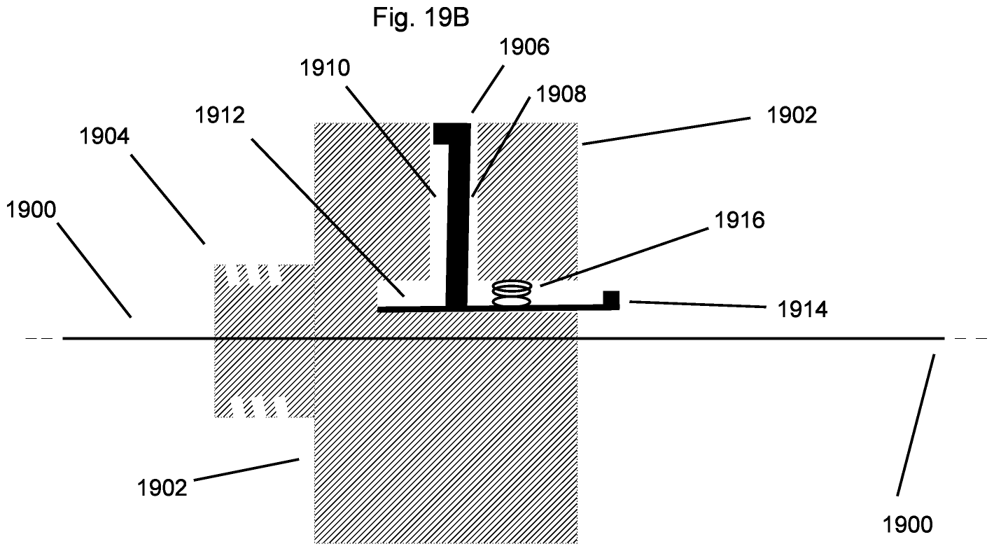

FIGS. 18A and 18B depict a side cross-section view of a needle apparatus comprising a safety mechanism incorporating two biased release arms. To assist understanding, spring 1812 is shown in perspective view in FIG. 18A and is removed from view in FIG. 18B. Needle 1800 is secured by hub 1802, which comprises hub adapter 1804. A first button arm 1808A is positioned in first button channel 1806A and is coupled to a first release arm 1810A, which is coupled to hub 1802 at or near one end of first release arm 1810A. At the opposite end of first release arm 1810A is first release mechanism 1820A. A second button arm 1808B is positioned in a second button channel 1806B and is coupled to a second release arm 1810B, which is coupled to hub 1802 at or near one end of second release arm 1810B. At the opposite end of second release arm 1810B is second release mechanism 1820B. When packaged for shipment and use, first release mechanism 1820A and second release mechanism 1820B are each biased so that first release mechanism 1820A and second release mechanism 1820B lock with cap retainer edge 1822, thereby holding inner cylinder 1818 in place. The bias may be obtained by the selection of material(s) for first release arm 1810A, second release arm 1810B, and hub 1802, and by the design of first release arm 1810A and the second release arm 1810B and their connections to hub 1802. A spring 812 is compressed in the position shown. In this position, inner cylinder 1818, middle cylinder 1816, and outer cylinder 1814 are held in place. When first button arm 1808A and second button arm 1808B are pressed, the inward forces move first release arm 1810A downward and second release arm 1810B upward, which moves first release mechanism 1820A downward and second release mechanism 1820B upward, until both are clear of cap retainer edge 1822. At that point, spring 812 expands towards its natural state, pushing inner cylinder 1818 to the right.

FIGS. 19A and 19B depict a side cross-section view of a needle apparatus comprising a safety mechanism incorporating a spring-biased release arm. Needle 1900 is secured by hub 1902, which comprises hub adapter 1904. A button 1906 is mounted on button arm 1908 which is positioned in button channel 1910 and is coupled to release arm 1912. Release arm 1912 is coupled to hub 1902 at or near one end of release arm 1912. At the opposite end of release arm 1912 is a release mechanism 1914. A bias spring 1916 is coupled to release arm 1912 and hub 1902 and, in the embodiment shown in FIGS. 19A and 19B, presses downward on release arm 1912. To operate the release mechanism, a user presses button 1906 forward until it has cleared the end of hub 1902 and can enter into button channel 1910. At that point, bias spring 1916 presses down on release arm 1912, pulling button arm 1908 down through button channel 1910 and pushing release mechanism 1914 down as well. The result is shown in FIG. 19B. This movement can release a safety cap mechanism.

In an alternative embodiment of the apparatus shown in FIGS. 19A and 19B, bias spring 1916 is biased to contract. In this embodiment, a user must push button 1906 forward until it has cleared the end of hub 1902 and can enter into button channel 1910. Because bias spring 1916 is biased to contract (or remain contracted), the user must further push button 1906 and button arm 1908 down into button channel 1910 in order to move release mechanism 1914.

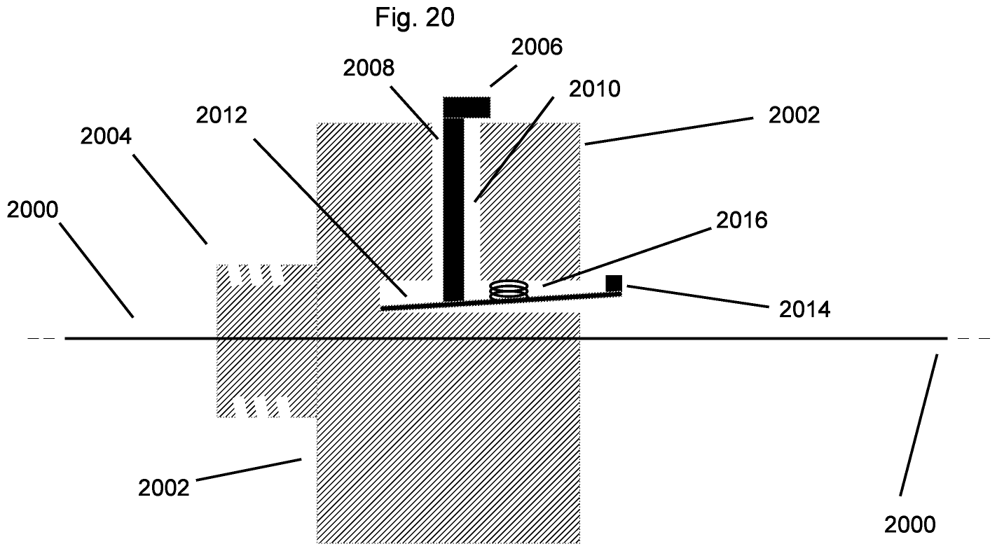

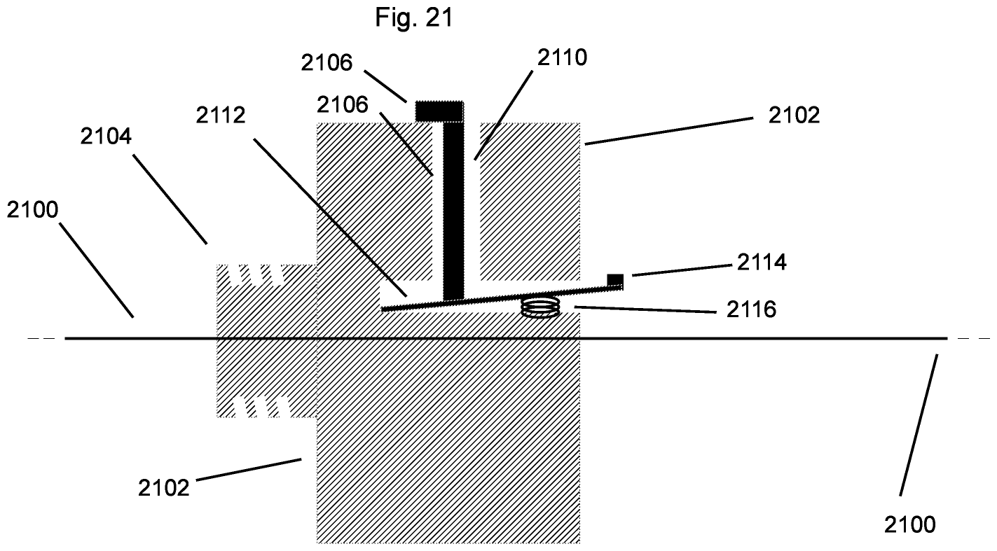

FIG. 20 depicts a side cross-section view of a needle apparatus comprising a safety mechanism incorporating another spring-biased release arm. Needle 2000 is secured by hub 2002, which comprises hub adapter 2004. A button 2006 is mounted on button arm 2008 which is positioned in button channel 2010 and is coupled to release arm 2012. Release arm 2012 is coupled to hub 2002 at or near one end of release arm 2012. At the opposite end of release arm 2012 is a release mechanism 2014. A bias spring 2016 is coupled to release arm 2012 and hub 2002 and, in the embodiment shown in FIG. 20, presses downward on release arm 2012. To operate the release mechanism, a user pulls button 2006 backward until it has cleared the end of hub 2002 and can enter into button channel 2010. At that point, bias spring 2016 presses down on release arm 2012, pulling button arm 2008 down through button channel 2010 and pushing release mechanism 2014 down as well. This movement can release a safety cap mechanism.

In an alternative embodiment of the apparatus shown in FIG. 20, bias spring 2016 is biased to contract. In this embodiment, a user must pull button 2006 backward until it has cleared the end of hub 2002 and can enter into button channel 2010. Because bias spring 2016 is biased to contract (or remain contracted), the user must further push button 2006 and button arm 2008 down into button channel 2010 in order to move release mechanism 2014.

FIG. 21 depicts a side cross-section view of a needle apparatus comprising a safety mechanism incorporating another spring-biased release arm. Needle 2100 is secured by hub 2102, which comprises hub adapter 2104. A button 2106 is mounted on button arm 2108 which is positioned in button channel 2110 and is coupled to release arm 2112. Release arm 2112 is coupled to hub 2102 at or near one end of release arm 2112. At the opposite end of release arm 2112 is a release mechanism 2114. A bias spring 2116 is coupled to release arm 2112 and hub 2102 and, in the embodiment shown in FIG. 21, pulls downward on release arm 2112. To operate the release mechanism, a user presses button 2106 forward until it has cleared the end of hub 2102 and can enter into button channel 2110. At that point, bias spring 2116 presses down on release arm 2112, pulling button arm 2108 down through button channel 2110 and pushing release mechanism 2114 down as well. This movement can release a safety cap mechanism.

In an alternative embodiment of the apparatus shown in FIG. 21, bias spring 2116 is biased to expand. In this embodiment, a user must push button 2106 forward until it has cleared the end of hub 2102 and can enter into button channel 2110. Because bias spring 2116 is biased to expand (or remain expanded), the user must further push button 2106 and button arm 2108 down into button channel 2110 in order to move release mechanism 2114.

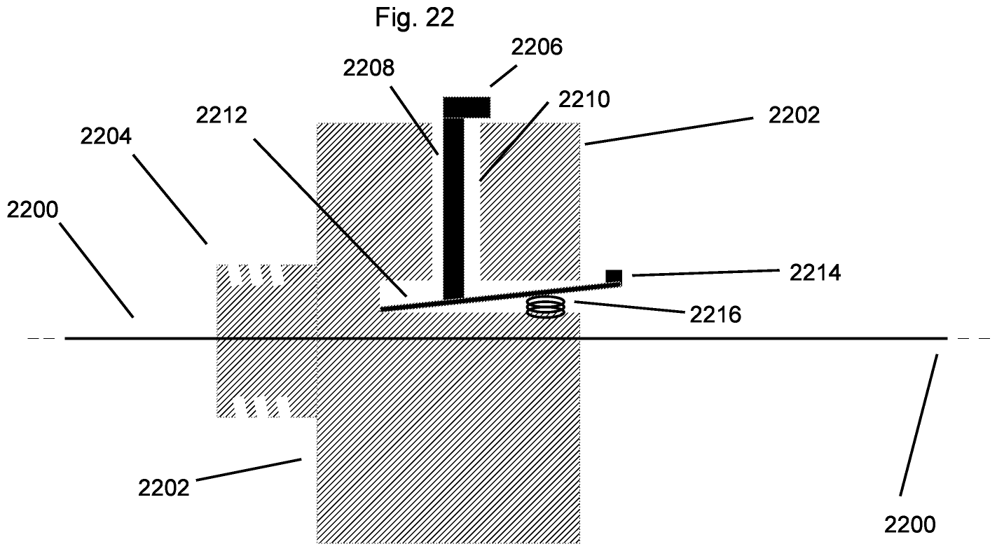

FIG. 22 depicts a side cross-section view of a needle apparatus comprising a safety mechanism incorporating another spring-biased release arm. Needle 2200 is secured by hub 2202, which comprises hub adapter 2204. A button 2206 is mounted on button arm 2208 which is positioned in button channel 2210 and is coupled to release arm 2212. Release arm 2212 is coupled to hub 2202 at or near one end of release arm 2212. At the opposite end of release arm 2212 is a release mechanism 2214. A bias spring 2216 is coupled to release arm 2212 and hub 2202 and, in the embodiment shown in FIG. 22, pulls down on release arm 2212. To operate the release mechanism, a user pulls button 2206 backward until it has cleared the end of hub 2202 and can enter into button channel 2210. At that point, bias spring 2216 pulls down on release arm 2212, pulling button arm 2208 down through button channel 2210 and pulling release mechanism 2214 down as well. This movement can release a safety cap mechanism.

In an alternative embodiment of the apparatus shown in FIG. 22, bias spring 2216 is biased to expand. In this embodiment, a user must pull button 2206 backward until it has cleared the end of hub 2202 and can enter into button channel 2210. Because bias spring 2216 is biased to expand (or remain expanded), the user must further push button 2206 and button arm 2208 down into button channel 2210 in order to move release mechanism 2214.

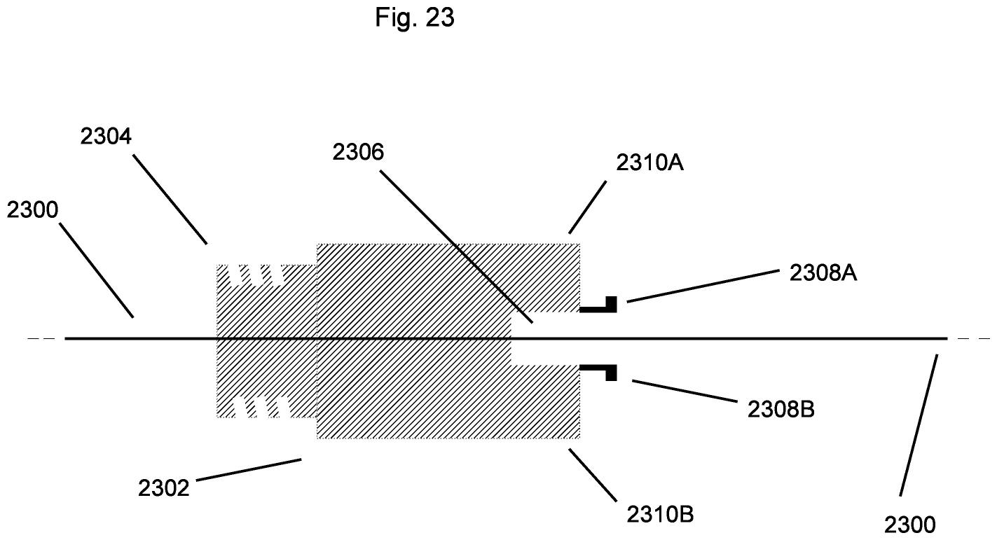

FIG. 23 depicts a side cross-section view of a needle apparatus comprising a safety mechanism incorporating a squeeze release. Needle 2300 is secured by hub 2302, which comprises hub adapter 2304. A cavity 2312 extends in from the front of hub 2302. A first release mechanism 2308A extends from hub 2302 adjacent to the cavity 2306. An optional second release mechanism 2308B extends from the hub 2302 adjacent to the cavity 2306 and opposite the first release mechanism 2308A. In order to engage the release mechanisms, a user squeezes the hub 2302 at between first squeeze point 2310A and second squeeze point 2310B. Squeezing hub 2302 at these points compresses the front of hub 2302 at the point of cavity 2306, thereby moving first release mechanism 2308A and optional second release mechanism 2308B inward and releasing a cap (not shown). First release mechanism 2308A and optional second release mechanism 2308B may be formed of the same material as hub 2302 or may be formed of a different material and coupled to hub 2302 during manufacturing.

FIG. 24A depicts a safety mechanism comprising a release frame to releasably secure a spring-loaded cap. Needle 2400 is secured by hub 2404, which is engaged with syringe 2402. Spring 2406 is secured at one end to hub 2404 and is secured at its opposite end to cap 2408. As shown, spring 2406 is in the compressed position and secured in place by release frame 2410. Specifically, a lip on cap 2408 is held back by an inside edge of release frame 2410. In order to release the spring 2406 and cap 2408, a user squeezes the side surfaces of release frame 2410, which deforms opening 2412 and disengages the lip of cap 2408 from the inside edge of release frame 2410.

FIG. 24B depicts a side view of spring 2406 and cap 2408 held in place by release frame 2410. Cap 2408 comprises lip 2414, which is held by release frame 2410. From this side view, a user may release cap 2408 and lip 2414 by compressing release frame 2410 in the vertical direction, for example, by pressing down on the top with a finger and pressing upward from the bottom with a thumb. In some embodiments, release frame 2410 can be made of metal, plastic, or other suitable material. FIG. 24C depicts a side view showing spring 2406 and cap 2408 in an extended position after release frame 2410 has been compressed and spring 2406 has been allowed to extend along the length of needle 2400.

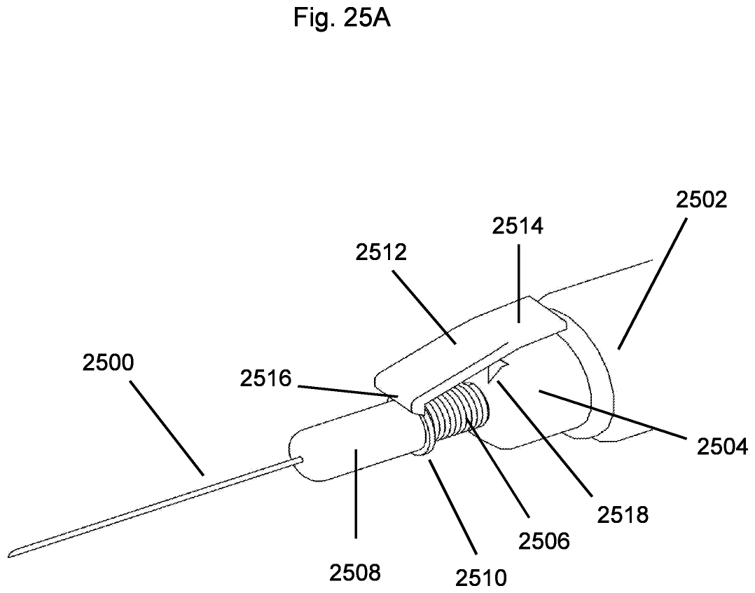

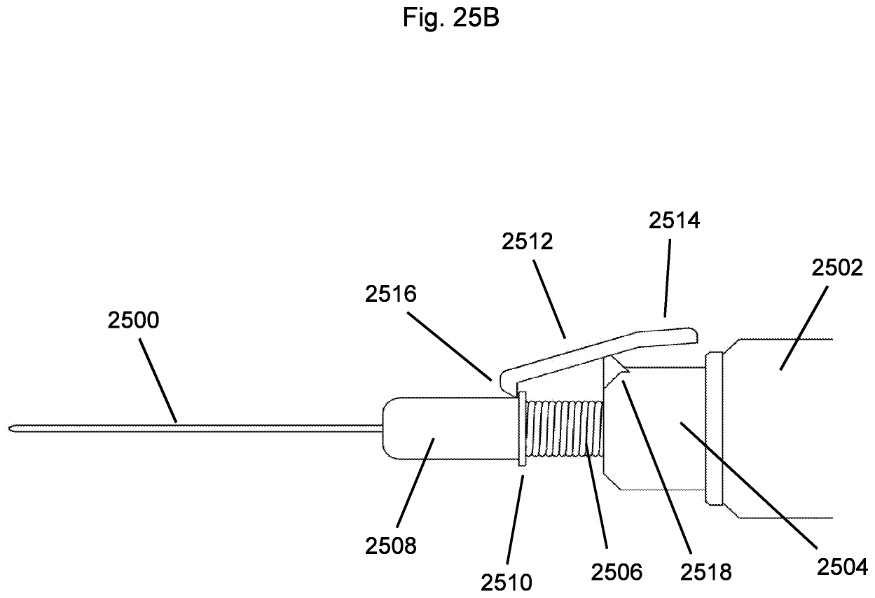

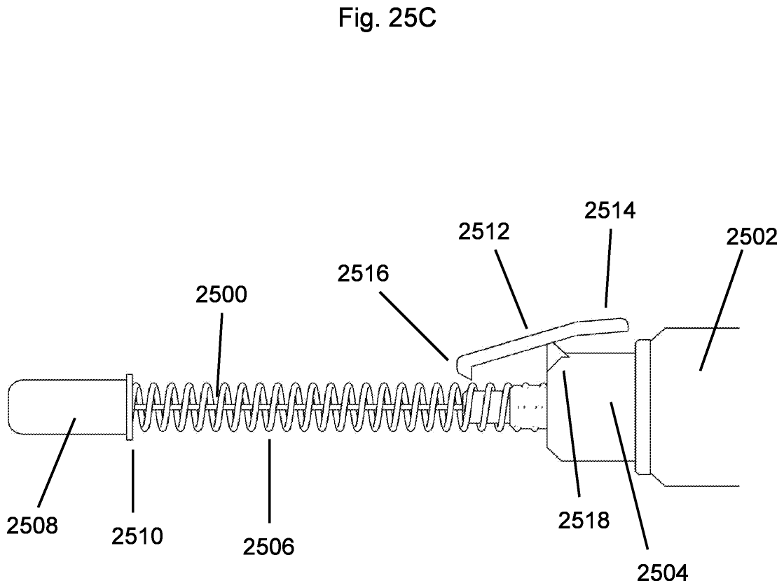

FIG. 25A depicts a safety mechanism comprising a lever to releasably secure a spring-loaded cap. Needle 2500 is secured by hub 2504, which is engaged with syringe 2502. Spring 2506 is secured at one end to hub 2504 and is secured at its opposite end to cap 2508. As shown, spring 2506 is in the compressed position and secured in place. Lip 2510 on cap 2508 is engaged with lip 2516 on lever 2512. At the opposite end of lever 2512 is button 2514. When pressed, button 2514 rotates lever 2512 about fulcrum 2518, which lifts lip 2516 out of contact with lip 2510. Spring 2506 may then extend, pushing cap 2508 along the length of needle 2500.

FIG. 25B depicts a side view showing the engagement of lip 2510 and lip 2516 when the spring is in the compressed position. In some embodiments, hub 2504, fulcrum 2518, lever 2512, button 2514, and lip 2516 comprise a unitary body manufactured of the same material. For example, the foregoing parts may be injected molded together. It is contemplated that, in some embodiments, spring 2506 and cap 2508 may also be injected molded, together and/or with hub 2504, fulcrum 2518, lever 2512, button 2514, and lip 2516.

FIG. 25C depicts a side view showing spring 2506 and cap 2508 in an extended position after button 2514 has been compressed, disengaging lip 2516 from lip 2510, and spring 2506 has been allowed to extend along the length of needle 2500. In some embodiments, a second lever with a fulcrum, lip, and button may be positioned on the hub. In such a configuration, a user would need to press two buttons to release the spring and cap, thereby providing an additional safety measure against accidental release of the spring and cap when the syringe is in use.

It should be understood that one or more features disclosed in any of the foregoing embodiments may be combined with one or more features disclosed in other embodiments. A non-exhaustive list of example combinations may be formed by: any or none of the caps described with reference to FIGS. 3A-3C, 6A-6D, 8A-8B, 9A-9B, 10A-10B, 11A-11B, and/or 12A-12B, in combination with any or none of the release mechanisms described with reference to FIGS. 3C, 5A-5B, 17A-17B, 18A-18B, 19A-19B, 20, 21, 22, and/or 23, in combination with any or none of the cylinder constructions described with reference to FIGS. 13A-13D, 14A-14C, and/or 15A-15C, optionally in combination with the sheath described with reference to FIG. 7, optionally in combination with the elongated hub described with reference to FIG. 16. Further, while the foregoing disclosure describes needles for use with reusable syringes, one or more of the principles disclosed herein may be used with single-use syringes as well.

The detailed description is not intended to be limiting or represent an exhaustive enumeration of the principles disclosed herein. It will be apparent to those of skill in the art that numerous changes may be made in such details without departing from the spirit of the principles disclosed herein.

What is claimed is:

1. A needle safety mechanism, comprising:
a needle;
a hub coupled to the needle, the hub comprising a threaded portion configured to be coupled to a syringe;
a spring coupled to the hub and expandable between a compressed position and an extended position along a length of the needle;
a cap coupled to the spring; and
a release mechanism coupled to the hub and releasably coupled to the cap.

2. The needle safety mechanism of claim 1, wherein the release mechanism comprises a release frame.

3. The needle safety mechanism of claim 2, wherein the release frame comprises an opening, and the safety cap comprises a lip configured to engage with the release frame.

4. The needle safety mechanism of claim 1, wherein the release mechanism comprises a lever, a fulcrum, a button portion, and a lip releasably engaged with the cap.

5. The needle safety mechanism of claim 4, wherein the release mechanism is configured so that a compression force on the button portion disengages the lip from the cap.

6. The needle safety mechanism of claim 1, wherein:
the cap comprises an end comprising a hole; and
the cap comprises a sidewall.

7. The needle safety mechanism of claim 6, wherein the cap comprises an interior protrusion.

8. The needle safety mechanism of claim 1, wherein the cap comprises a safety flap.

9. The needle safety mechanism of claim 1, wherein release mechanism comprises a first arm coupled to a biased second arm, the second arm comprising a lip releaseably engaged with the cap.

10. The needle safety mechanism of claim 1, comprising one or more telescoping caps.

11. A needle safety mechanism, comprising:
a needle;
a hub coupled to the needle, the hub comprising a threaded portion configured to be coupled to a syringe;
a spring coupled to the hub and expandable between a compressed position and an extended position along a length of the needle;
a cap coupled to the spring; and
wherein the hub comprises a release mechanism configured to release the spring from the compressed position to the extended position.

12. The needle safety mechanism of claim 11, wherein:
the release mechanism comprises a cavity and at least one release hook releaseably engaged with the cap; and
the release mechanism is configured so that a compression force against the cavity disengages the at least one release hook from the cap.

13. The needle safety mechanism of claim 11, wherein:
the cap comprises an end comprising a hole; and
the cap comprises a sidewall.

14. The needle safety mechanism of claim 13, wherein the cap comprises an interior protrusion.

15. The needle safety mechanism of claim 11, wherein the cap comprises a safety flap.

16. The needle safety mechanism of claim 11, wherein release mechanism comprises a first arm coupled to a biased second arm, the second arm comprising a lip releaseably engaged with the cap.

17. The needle safety mechanism of claim 11, comprising one or more telescoping caps.
